(19) United States
(12) Patent Application Publication
(10) Pub. No.: US 2025/0146913 A1
(43) Pub. Date: May 8, 2025

(54) DOUBLE DOMED FLOAT

(71) Applicant: Hanuman Pelican, Inc., New Orleans, LA (US)

(72) Inventors: Randy DORIAN, San Diego, CA (US); Michael D. LEACH, Warsaw, IN (US); Richard W. STORRS, Berkeley, CA (US); Scott R. KING, New Orleans, LA (US)

(73) Assignee: Hanuman Pelican, Inc., New Orleans, LA (US)

(21) Appl. No.: 18/829,802

(22) Filed: Sep. 10, 2024

Related U.S. Application Data
(60) Provisional application No. 63/596,908, filed on Nov. 7, 2023.

(51) Int. Cl. G01N 1/34

(52) U.S. Cl. CPC G01N 1/34

(57) ABSTRACT

Double domed float apparatus and methods of use are described herein. The float may generally include a cylindrical portion with an upper portion and a lower portion each having a domed or hemispherical configuration and extending from opposite ends of the cylindrical portion. The lower portion may be configured to correspond in shape to the curved surface of the bottom portion of a tube. The density of the float may be configured to position the cylindrical portion, at equilibrium, below a layer of platelet-rich plasma such that the platelet-rich plasma is retained between the upper portion and an interior surface of the lumen.

### CROSS-REFERENCE TO RELATED APPLICATIONS

This application claims the benefits of priority to U.S. Prov. App. 63/596,908 filed Nov. 7, 2023, which is incorporated herein by reference in its entirety.

### FIELD OF THE INVENTION

The present invention relates to apparatus and methods for separating blood components. More particularly, the present invention relates to a foam collared float for effectively separating and removing specific components from blood.

### BACKGROUND OF THE INVENTION

Blood may be fractionated and the different fractions of the blood used for different medical needs. For instance, anemia (low erythrocyte levels) may be treated with infusions of erythrocytes. Thrombocytopenia (low thrombocyte (platelet) levels) may be treated with infusions of platelet concentrate.

The sedimentation of the various blood cells and plasma is based on the different specific gravity of the cells and the viscosity of the medium. When sedimented to equilibrium, the component with the highest specific gravity (density) eventually sediments to the bottom, and the lightest rises to the top. Under the influence of gravity or centrifugal force, blood spontaneously sediments into three layers. At equilibrium the top, low-density layer is a straw-colored clear fluid called plasma. Plasma is a water solution of salts, metabolites, peptides, and many proteins ranging from small (insulin) to very large (complement components). Plasma per se has limited use in medicine but may be further fractionated to yield proteins used, for instance, to treat hemophilia (factor VIII) or as a hemostatic agent (fibrinogen). The term platelet rich plasma (PRP) is used for this component because most of the plasma proteins and platelets in the whole blood are in the plasma following slow centrifugation so the concentration of platelets in the plasma has increased while suspended in supernatant plasma. The uppermost layer after centrifugation typically contains plasma proteins only and is typically called platelet-poor plasma (PPP) due to the absence or low number of platelets as a result of a “hard spin”.

The bottom, high-density layer is a deep red viscous fluid comprising a nuclear red blood cells (RBC) specialized for oxygen transport. The red color is imparted by a high concentration of chelated iron or heme that is responsible for the erythrocytes high specific gravity. Packed erythrocytes, matched for blood type, are useful for treatment of anemia caused by, e.g., bleeding. The relative volume of whole blood that consists of erythrocytes is called the hematocrit, and in normal human beings can range from about 38% to about 54%.

The intermediate layer is the smallest layer, appearing as a thin white band on top the erythrocyte layer and below the plasma, and is called the buffy coat. The buffy coat itself has two major components, nucleated leukocytes (white blood cells) and anuclear smaller bodies called platelets (or thrombocytes). Leukocytes confer immunity and contribute to debris scavenging. Platelets seal ruptures in the blood vessels to stop bleeding and deliver growth and wound healing factors to the wound site. The buffy coat may be separated from whole blood when the blood is subjected to a “hard spin” in which the whole blood is spun hard enough and long enough so that platelets sediment from plasma onto packed red cells and white cells percolate up through red cell pack to the interface between red cells and plasma.

When whole blood is centrifuged at a low speed (e.g., up to 1,000 g) for a short time (e.g., two to four minutes) white cells sediment faster than red cells and both sediment much faster than platelets. At higher speeds the same distribution is obtained in a shorter time. The method of harvesting PRP from whole blood is based on this principle. Centrifugal sedimentation that takes the fractionation only as far as separation into packed erythrocytes and PRP is called a “soft spin” which is typically used to describe centrifugation conditions under which erythrocytes are sedimented but platelets remain in suspension. “Hard spin” is typically used to describe centrifugation conditions under which erythrocytes sediment and platelets sediment in a layer immediately above the layer of erythrocytes.

The auto-transfusion equipment used to make autologous platelet concentrates requires a skilled operator and considerable time and expense and these devices require a large prime volume of blood. While many of these devices have somewhat reduced the cost and the time required, skilled operators and time are still required. Accordingly, there remains a need for simple and effective methods and devices for separating and removing components from whole blood.

### SUMMARY OF THE INVENTION

Double domed float apparatus may generally include a cylindrical portion with an upper portion and a lower portion each having a domed or hemispherical configuration and extending from opposite ends of the cylindrical portion. The lower portion may be configured to correspond in shape to the curved surface of the bottom portion of a tube. The density of the float may be configured to position the cylindrical portion, at equilibrium, below a layer of platelet-rich plasma such that the platelet-rich plasma is retained between the upper portion and an interior surface of the lumen.

The features and embodiments described in relation to the double domed embodiments may be combined in any number of additional combinations with any of the features described herein and are intended to be within the scope of the present description. Details of additional features for combination are further described in U.S. patent application Ser. No. 18/320,702 filed May 19, 2023 which is incorporated herein by reference in its entirety.

One variation may include a foam collared float which may have a wide float stem portion. As described herein, the foam is defined as a closed cell foam material in which the individual cells (or at least a majority of the individual cells) of the foam material are enclosed. A narrow float stem portion may be fixedly attached to the wide float stem portion. A float hole may have a first float hole end on the wide float stem portion and may have a second float hole end on the narrow float stem portion. A foam collar may be fixedly or detachably connected to the narrow float stem portion. In other variations, the foam collar may instead be positioned along the wide float stem portion while seated into secure placement within a circumferential groove defined around the float.

The wide float stem portion may have an upper float end located on a proximal end of the wide float stem portion. The narrow float stem portion may have a lower float end located on a distal end of the narrow float stem portion. The upper float end may be concave. The lower float end may be concave. The float hole may run through the center of the wide float stem portion and the center of the narrow float stem portion. The wide float stem portion may have a wide float stem portion diameter around 0.758 inches and may have a wide float stem portion length around 0.450 inches. The narrow float stem portion may have a narrow float stem portion diameter around 0.635 inches and may have a narrow float stem portion length around 0.250 inches.

The foam collar may have a foam collar inner dimension around 0.375 inches and may have a foam collar outer dimension around 0.625 inches. The foam collar may have an uncompressed density around 0.33 grams per cubic centimeter. The foam collar may have a fully compressed density between 0.95 and 1.20 grams per cubic centimeter.

One variation may include a foam collared float which may have a float stem. The float stem may have a float stem distal end and a float stem proximal end. The foam collared float may have a float hole. The float hole may have a first float hole end on the float stem distal end and may have a second float hole end on the float stem proximal end. The foam collared float may have a foam collar. The foam collar may be fixedly or detachably connected to the float stem.

The float stem distal end may be concave. The float stem proximal end may be concave. The float hole may run through the center of the float stem. The float stem may have a float stem diameter around 0.635 inches and may have a float stem length around 0.250 inches. The foam collar may have a foam collar inner dimension around 0.375 inches and may have a foam collar outer dimension around 0.625 inches. The foam collar may have an uncompressed density around 0.33 grams per cubic centimeter. The foam collar may have a fully compressed density between 0.95 and 1.20 grams per cubic centimeter.

In one variation, the float may generally comprise a first float stem portion having a first diameter, a second float stem portion fixedly attached to the wide float stem portion and having a second diameter which is less than the first diameter, a channel having a first opening on the first float stem portion and a second opening on the second float stem portion, and a compressible collar positioned about the second float stem portion and having a third diameter which is greater than the first diameter.

In another variation, the float may generally comprise a float stem including a float stem distal end and a float stem proximal end, a float channel having a first float opening on the float stem distal end and a second float opening on the float stem proximal end, and a compressible collar fixedly or detachably connected about the float stem.

In another variation, the float may generally comprise a first float stem portion having a first diameter, a second float stem portion fixedly attached to the wide float stem portion and having a second diameter which is less than the first diameter, a channel having a first opening on the first float stem portion and a second opening on the second float stem portion, and a compressible collar positioned about the second float stem portion and having a third diameter which is greater than the first diameter.

In another variation, the float may generally comprise a float stem including a float stem distal end and a float stem proximal end, a float channel having a first float opening on the float stem distal end and a second float opening on the float stem proximal end, and a compressible collar detachably connected about the float stem.

In another variation, a method of securing a float within a container may generally comprise positioning a float into a bottom of a container, introducing a volume of fluid within the bottom of the container such that the fluid covers the float, and forming a vapor lock between the float and the container such that a position of the float is secured relative to an interior of the container.

In another variation, a method of separating blood may generally comprise introducing a volume of blood into a tube defining a lumen and a bottom end having a curved surface, applying a force to the blood contained within the tube and to a float positioned within the lumen, wherein the float has a cylindrical portion with an upper portion and a lower portion each having a domed or hemispherical configuration and extending from opposite ends of the cylindrical portion, and wherein the lower portion is configured to correspond in shape to the curved surface of the tube. The force may be further applied until the float reaches a position of equilibrium within the tube such that the cylindrical portion is positioned below a layer of platelet-rich plasma which is retained between the upper portion and an interior surface of the lumen.

In another variation of the float retention assembly, the float may generally comprise a tube defining a lumen and a bottom end having a curved surface, a float having a cylindrical portion with an upper portion and a lower portion each having a domed or hemispherical configuration and extending from opposite ends of the cylindrical portion, wherein the lower portion is configured to correspond in shape to the curved surface of the tube. The density of the float may be configured to position the cylindrical portion, at equilibrium, below a layer of platelet-rich plasma such that the platelet-rich plasma is retained between the upper portion and an interior surface of the lumen.

The cylindrical portion may also define a first portion having a first diameter and a second portion having a second diameter where the second portion is adjacent to the first portion. The first diameter may be less than the second diameter such that a step or shoulder is defined between the first portion and the second portion. Alternatively, a radius of the upper portion may be less than a radius of the cylindrical portion such that a step or shoulder is defined between the upper portion and the cylindrical portion.

### BRIEF DESCRIPTION OF THE DRAWINGS

FIG. 1A shows a side view of a foam collared float.

FIG. 1B shows a side view of a foam collared float positioned within a container having a sample to be processed.

FIGS. 1C and 1D show side and bottom views of another variation of the float having a domed or rounded distal portion.

FIG. 1E shows a side view of a float securely maintained in position at or near the bottom of the container.

FIG. 1F shows a side view of a float at an equilibrium position within the container after a sample has been processed.

FIG. 1G shows an example of an O-ring float collar having a circular cross-sectional shape when in its uncompressed configuration.

FIG. 1H shows another variation of a float collar having a rectangular shape also defining a hollow interior.

FIGS. 1I to 1P show various perspective views of several different float collar configurations having one or more select surfaces presenting a smooth and/or rough surface.

FIGS. 2A to 2E show side and perspective views of float variations which may be used with or without a float collar.

FIG. 2F shows a partial cross-sectional side view of the float variation of FIG. 2E illustrating the angled surfaces of the upper float portion.

FIG. 3 shows a side view of a foam collared float with a partial vapor lock.

FIGS. 4A to 4C illustrate side views of another float variation which utilize the vapor lock securement for different float sizes.

FIGS. 5A and 5B show side view of float secured within a container via a vapor lock securement.

FIG. 6 shows a side view of a variation of a foam collared float at equilibrium after centrifuging in an aqueous sucrose solution in a container tube.

FIG. 7 shows a perspective view of a foam collared float mounted upon a float stem.

FIG. 8 shows a perspective view of a foam collared float maintained within a container tube via the float collared float.

FIG. 9 shows a side view of a container after processing a volume of whole blood.

FIGS. 10A and 10B show side and top views, respectively, of yet another variation of the float configured to have a dome or hemispherical shape.

FIGS. 11A and 11B show side views of alternative variations of the float defining a step or shoulder between a first and second cylindrically-shaped portions.

FIGS. 11C to 11D also show top and bottom views, respectively, of the float defining the step or shoulder between the first and second cylindrically-shaped portions.

FIGS. 12A and 12B show side views of the various floats within a tube and the blood components separated relative to the position of the float at equilibrium.

FIGS. 13A to 13C illustrate one variation of a float secured within a bottom of the tube and how the float may move to its equilibrium position.

FIG. 14 illustrates an example of a packaging which defines a receiving channel or pocket formed for retaining the tube and float within.

FIG. 15 shows a side view of another example of the separated blood components after centrifugation.

### DETAILED DESCRIPTION OF THE INVENTION

Throughout the description, terms such as “top”, “above, “bottom”, “below” are used to provide context with respect to the relative positioning of components when, e.g., a container tube with fractional components of blood are positioned when the longitudinal axis of a container tube is positioned upright or non-horizontally. Such description is used for illustrative purposes only.

As discussed herein, when sedimented to equilibrium, the component with the highest specific gravity (density) eventually sediments to the bottom, and the lightest rises to the top. Under the influence of gravity or centrifugal force, blood spontaneously sediments into three layers. At equilibrium the top, low-density layer is a straw-colored clear fluid called plasma. The term platelet-rich plasma (PRP) is used for this component because most of the plasma proteins and platelets in the whole blood are in the plasma following slow centrifugation so the concentration of platelets in the plasma has increased while suspended in supernatant plasma. The bottom, high-density layer comprises sedimented red blood cells (RBC). The intermediate layer, if the blood is subjected to further centrifugation, is called the buffy coat.

The present invention relates to apparatus and methods for rapid fractionation of blood into its different components, e.g., erythrocyte, plasma, and platelet or buffy coat fractions. The design described herein for a buffy coat concentrator should provide platelet and white blood cell (WBC) yields comparable to other gravitational platelet separation (GPS) designs. The manufacturing costs should be lower and the devices are easy to use. It also allows for the user to choose a desired level of buffy coat concentration. Markings on the tube can be provided to indicate the amount of platelet-depleted plasma (PPP) to be withdrawn prior to resuspension of the buffy coat to yield a desired concentration factor (the more PPP removed, the higher the concentration after resuspension in the remaining volume). Because platelets sediment onto a thin layer of red blood cells (RBC) trapped within the upper float concavity, platelet damage should be minimal and resuspension should be easier than when platelets are sedimented directly onto a hard surface.

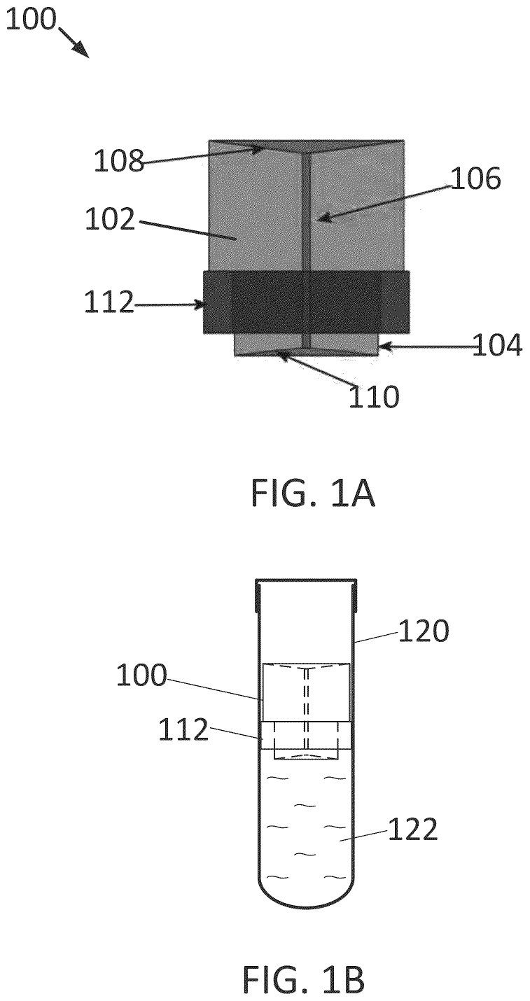

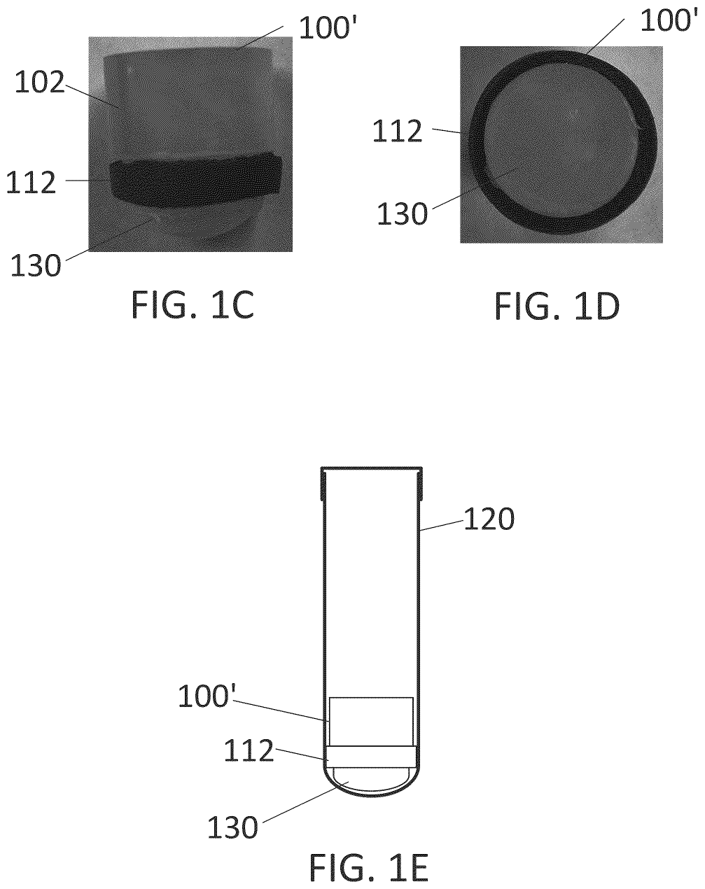

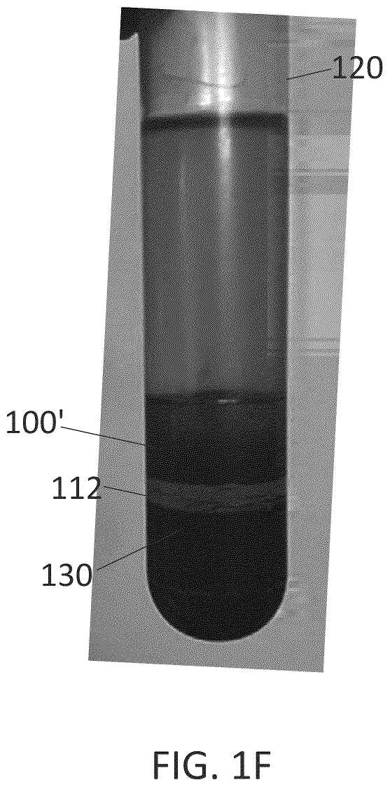

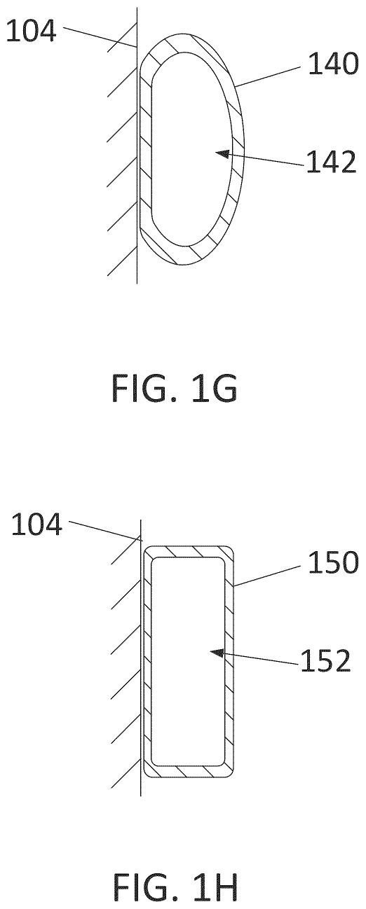

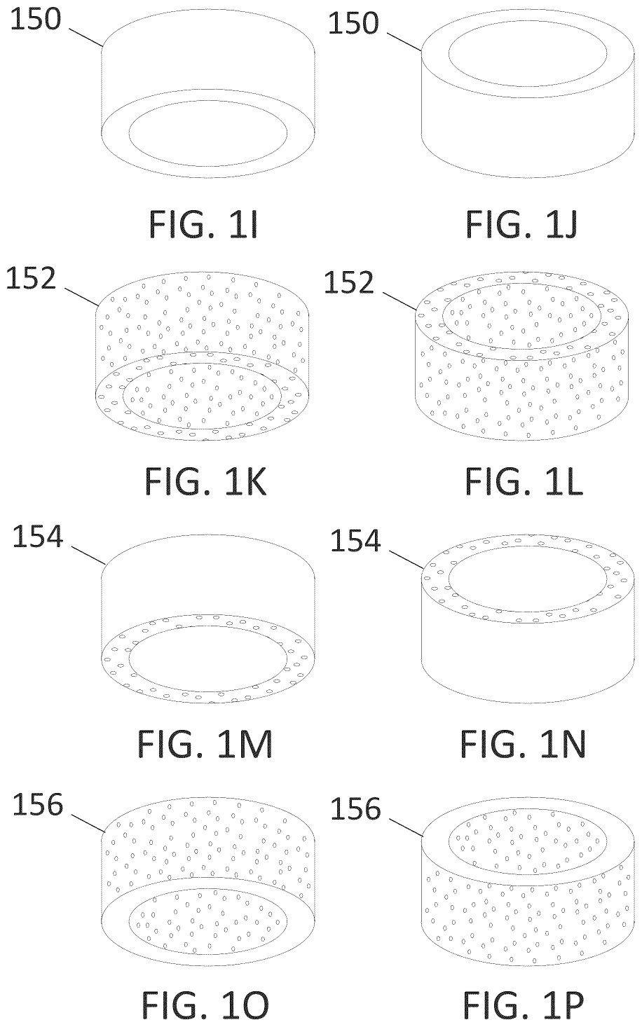

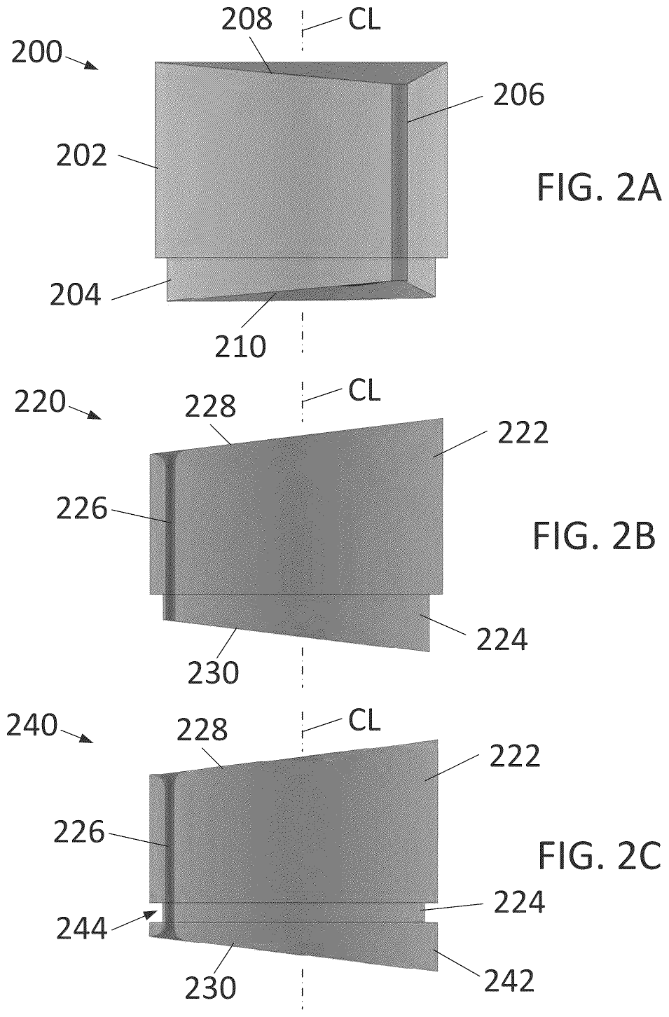

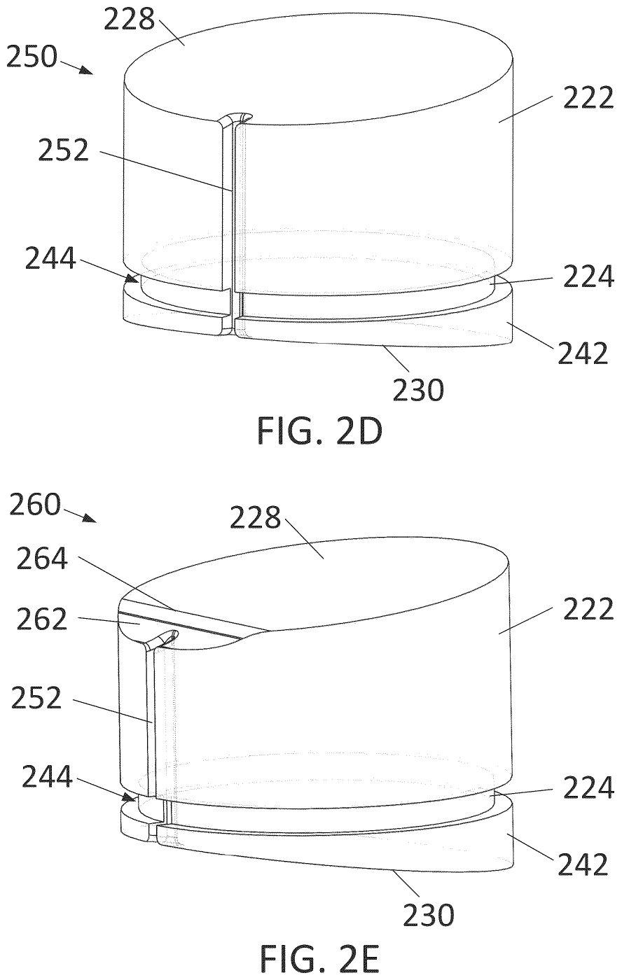

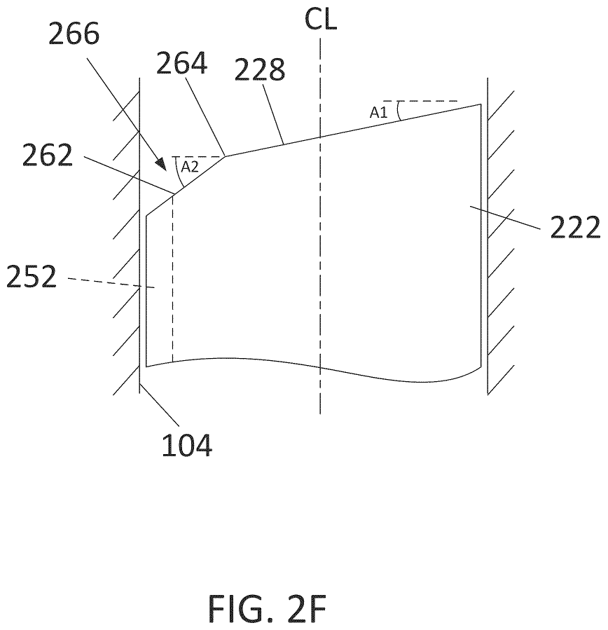

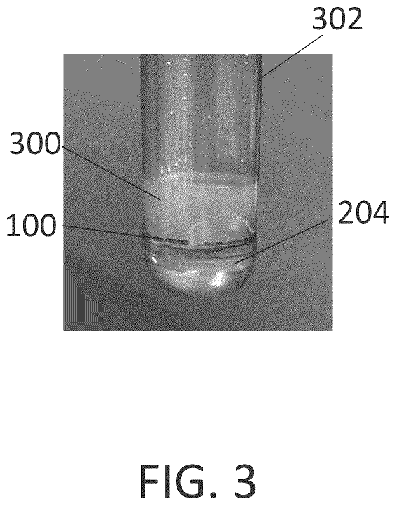

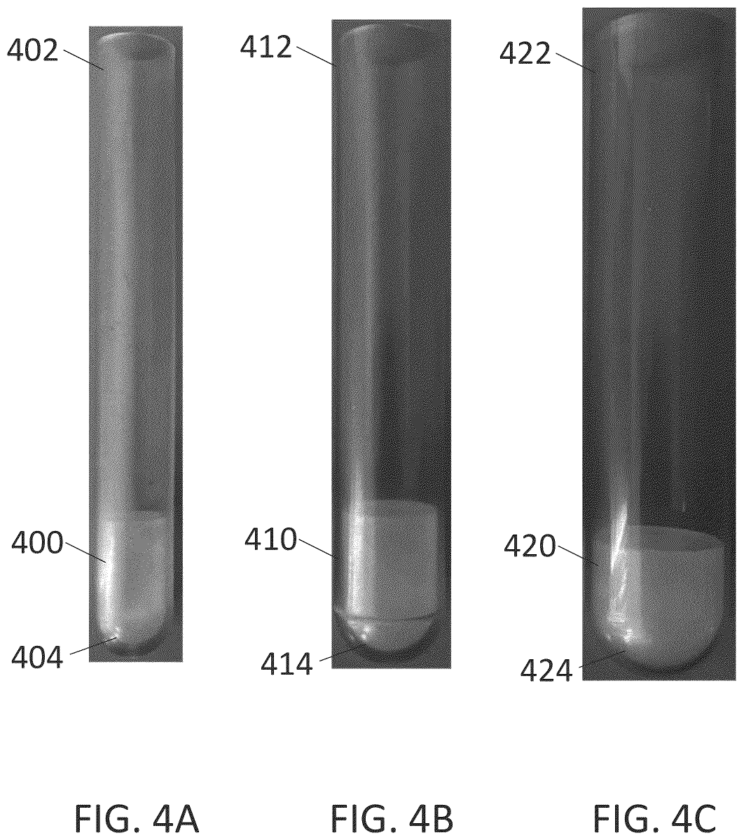

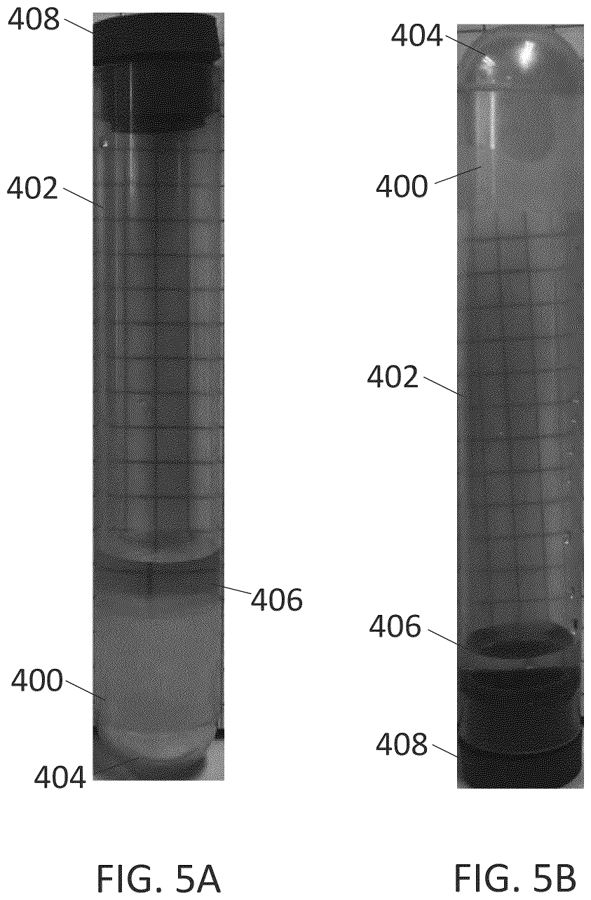

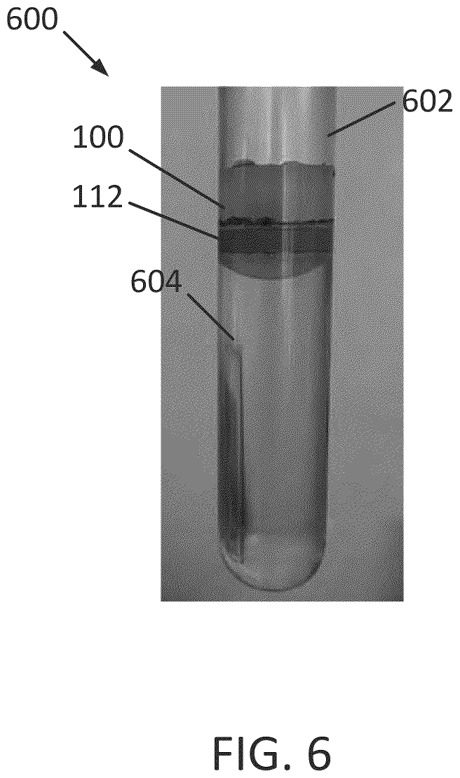

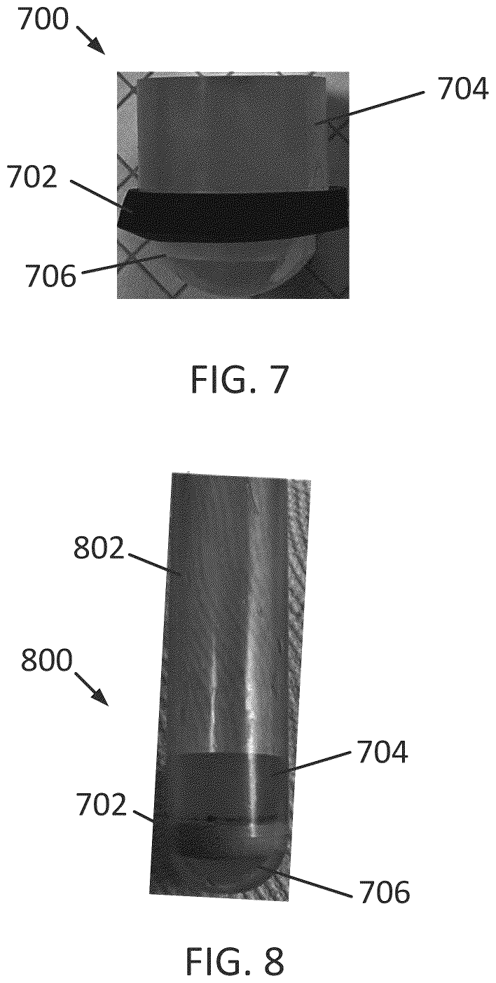

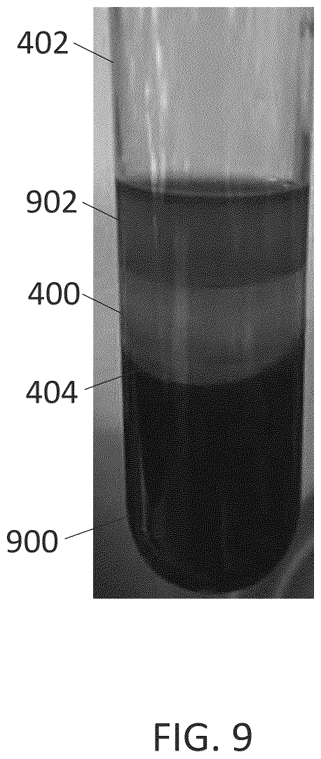

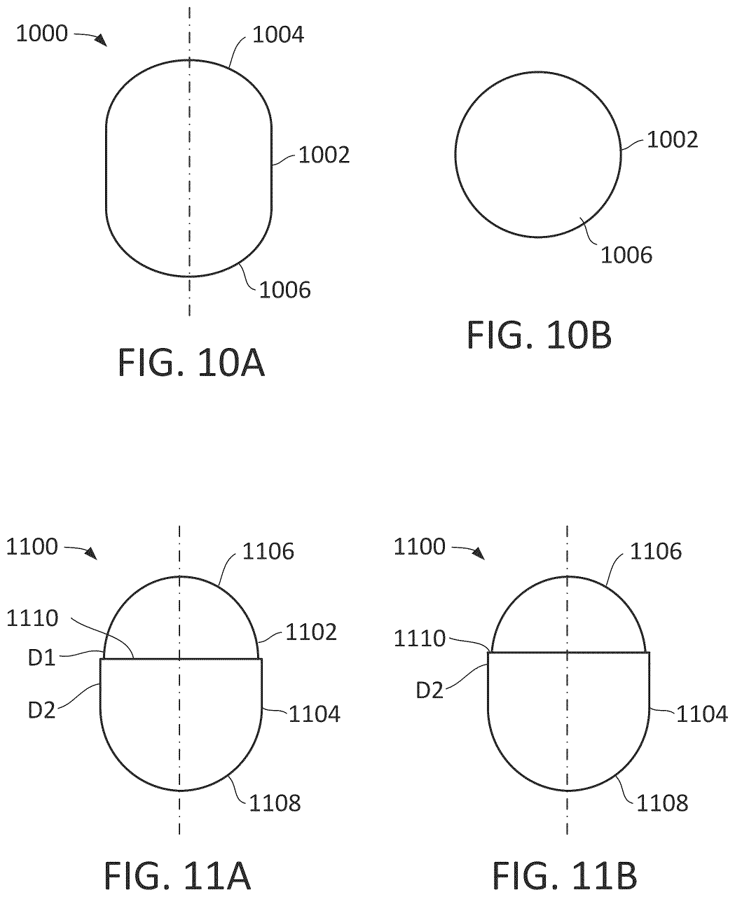

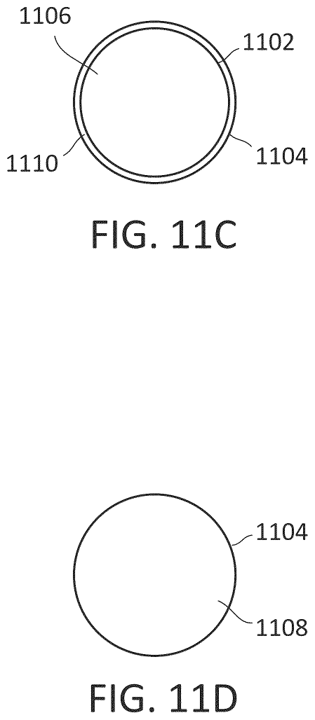

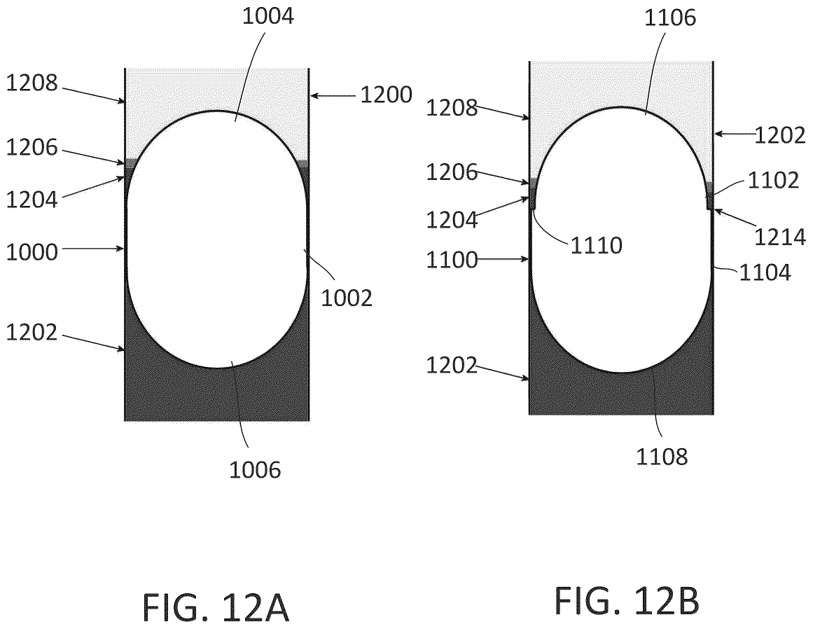

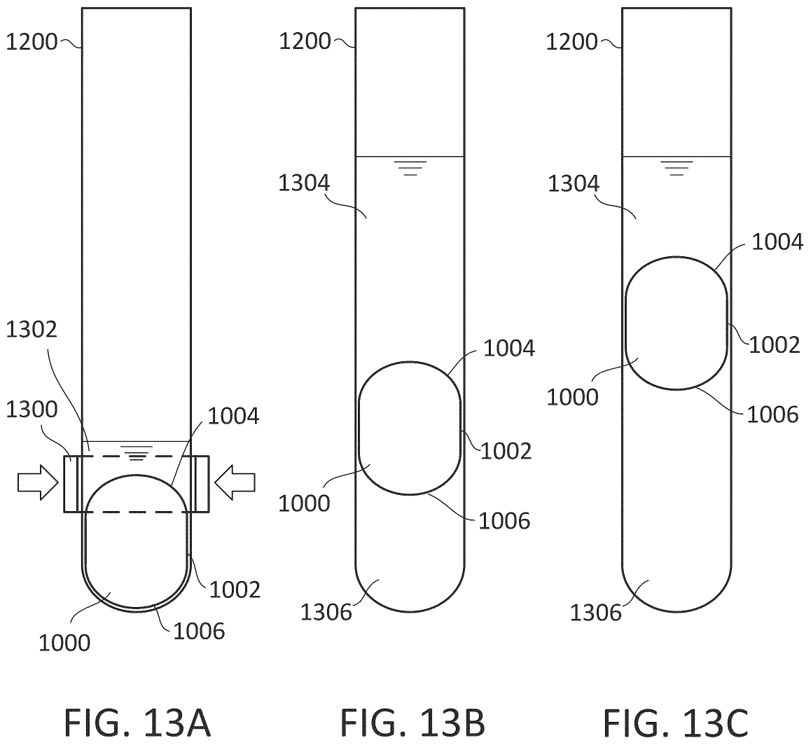

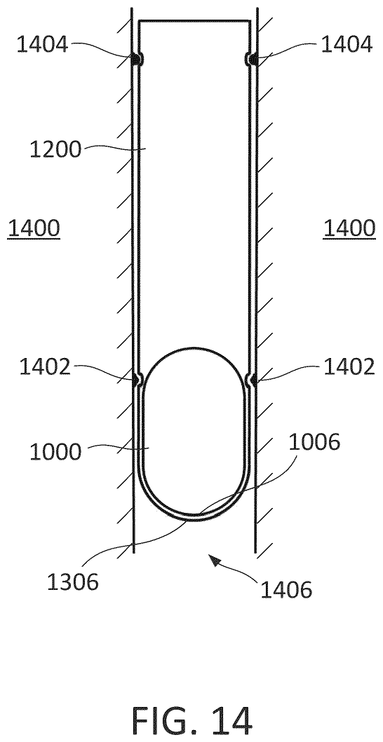

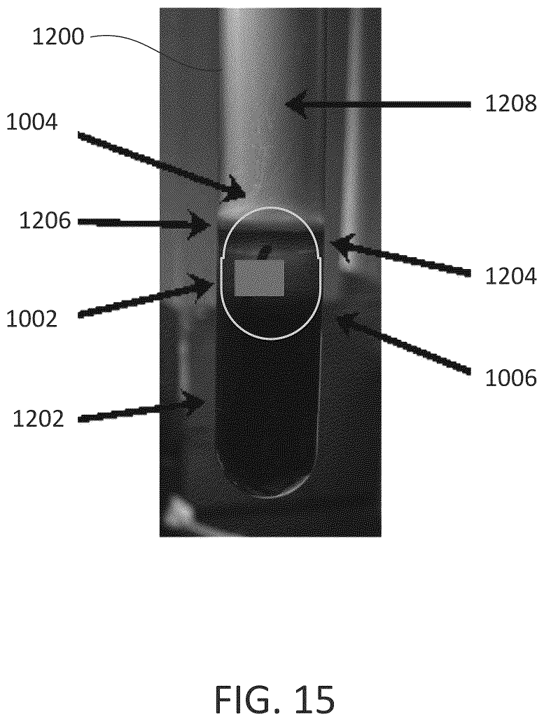

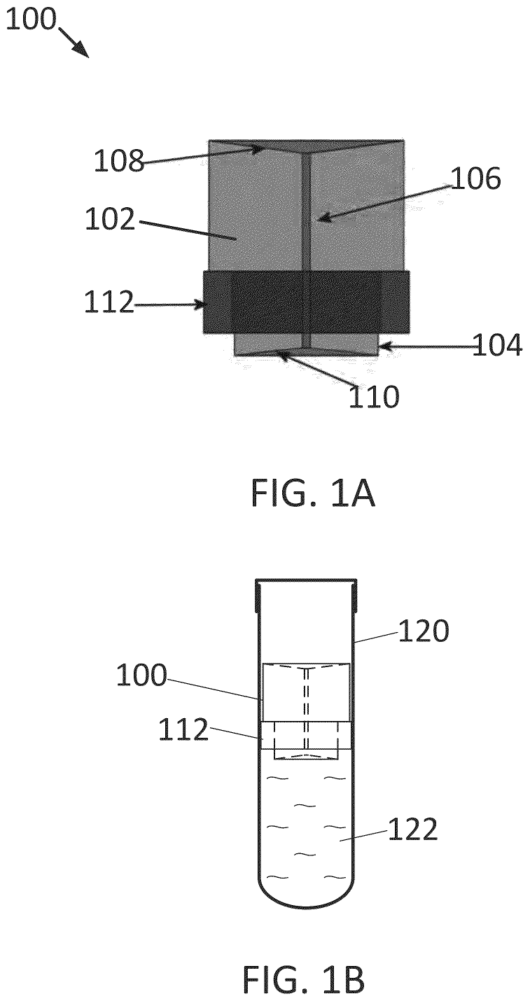

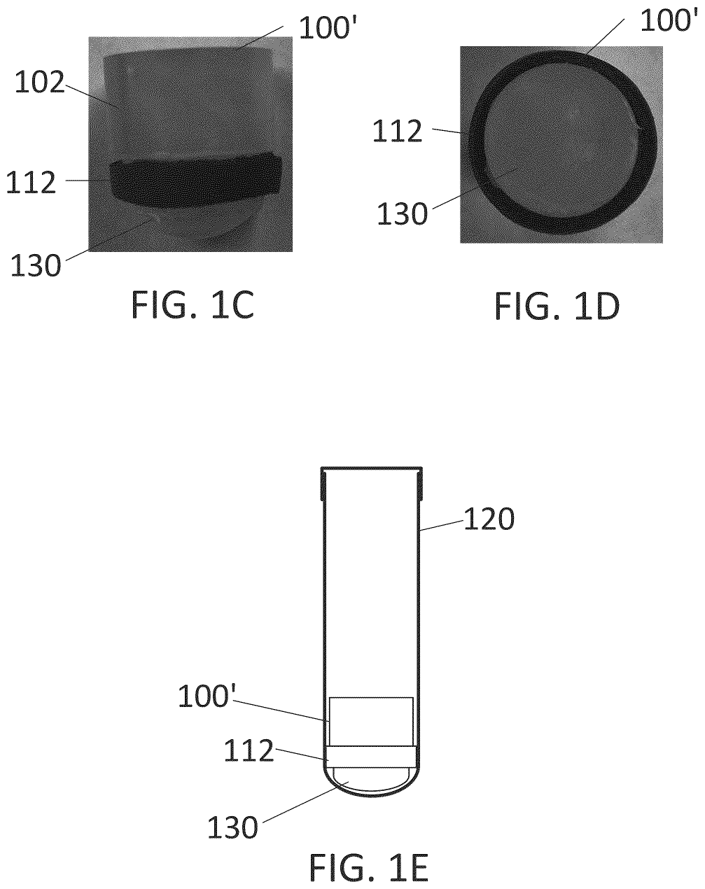

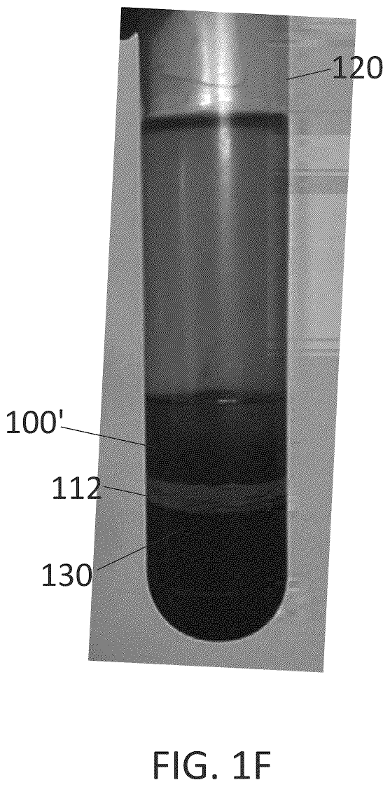

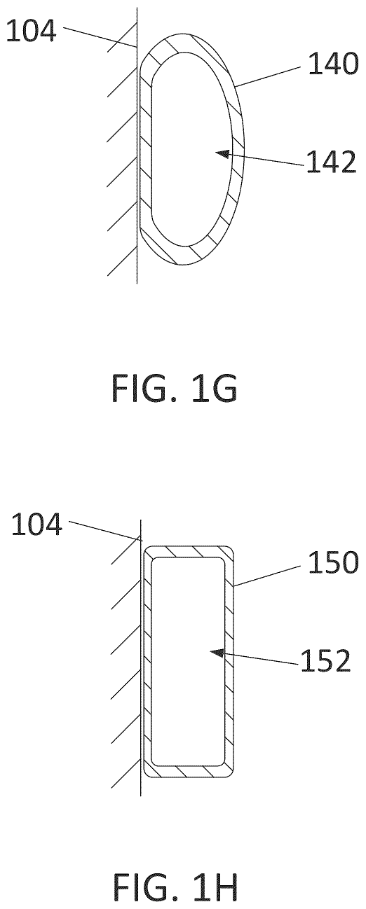

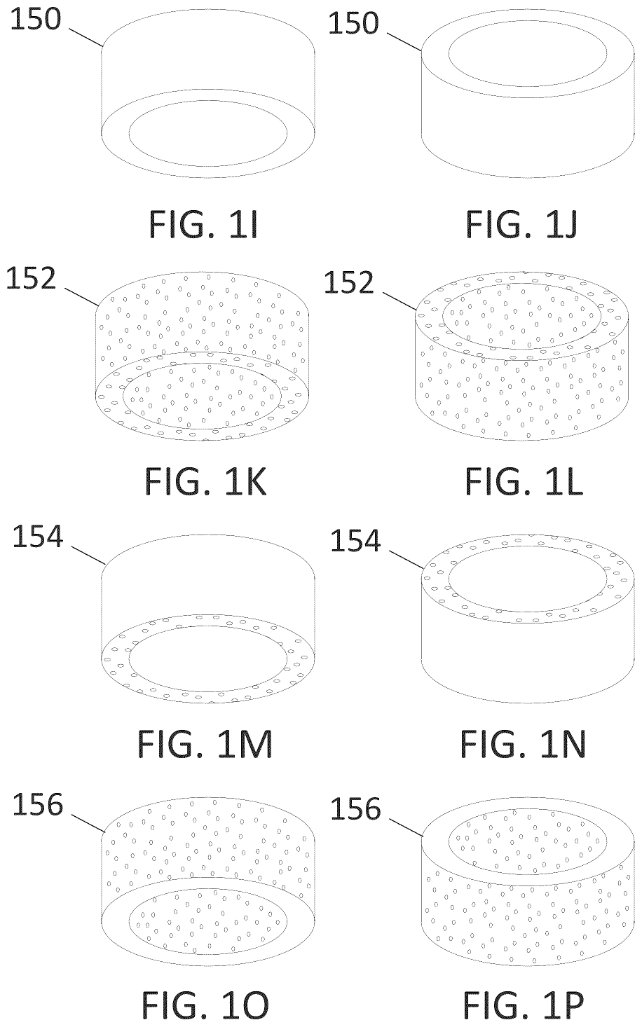

FIG. 1A and the illustrated step-by-step description herein are sufficient to permit appreciation of the principle and method of operation. Some variants described may also be useful for certain applications/markets and can allow also for preparation of WBC-reduced and RBC-reduced platelet-rich plasma (PRP).

One variation of the foam collar float 100 is shown in FIG. 1A as having a first float stem portion 102 with a first diameter and a second float stem portion 104 having a second diameter which is less than the first diameter and where the second float stem portion 104 is fixedly attached to the first float stem portion and extends in an opposite direction. As shown, the first float stem portion 102 may be formed as a cylindrically-shaped structure having a height a relatively wider diameter than the second float stem portion 104. The second float stem portion 104 may also have a cylindrically-shaped structure having a height although in other variations, the second float stem portion 104 may be shaped into other structures such as a domed-shaped structure or the second float stem portion 104 having a rounded distal portion. As described herein, the foam is defined as a closed cell foam material in which the individual cells (or at least a majority of the individual cells) of the foam material are enclosed.

While the cross-sectional shape of the first float stem portion 102 and the second float stem portion 104 may be uniform in shape, e.g., rounded or circular, both the first float stem portion 102 and the second float stem portion 104 may have shapes other than rounded, e.g., oval, square, rectangular, triangular, quad-angular, etc. Furthermore, while the shape of both the first float stem portion 102 and second float stem portion 104 may be uniform, e.g., both circular, they may be non-uniform as well where the first float stem portion 102 may have a first cross-sectional shape while the second float stem portion 104 may have a second cross-sectional shape different from the first cross-sectional shape.

A float channel 106 is defined and shown as running from an upper float portion 108 to a lower float portion 110 where the float channel 106 can extend from the center of the upper float portion 108 to a lower float portion 110. In other variations, the float channel 106 may be defined through the float 100 along locations other than the center of the upper float portion 108 and the center of the lower float portion 110. The upper float portion 108 may be defined as a surface which is shown as being concave in one variation. In other variations, the upper float portion 108 may be convex, flat, slanted, curved, or any combination thereof. The upper float portion 108 may be tapered to present a sloped conical shape or a sloped surface angled from one side of the upper float portion 108 towards the opposite side of the upper float portion 108. The lower float portion 110 is shown as being concave and having an angle relative to a longitudinal axis of the float collar 100. In other variations, the lower float portion 110 may be convex, flat, slanted, curved, or any combination thereof. The lower float portion 110 may be tapered to present a sloped conical shape or a sloped surface angled from one side of the lower float portion 110 towards the opposite side of the lower float portion 110. In any of these variations where the upper float portion 108 is concave, flattened, or convex, a layer may be applied to the upper float portion 108 which is relatively slippery. In one variation, a silicone layer may be formed upon the upper float portion 108 to facilitate the removal of platelets from the upper float portion 108.

While the lower float portion 110 may have any variation of surface, one advantage of a concave surface includes having white cells as well as platelets within a sample 122 positioned beneath the float 100 within a container 120 (as shown in FIG. 1B) being able to funnel up towards the buffy coat located along the upper float portion 108, e.g., within a concavity within the upper float portion 108, when the float 100 and container is spun for processing. This may help to improve recovery of white cells. Alternatively, a convexity of the lower surface may reduce the level of white cells.

A float collar 112 is shown as detachably connected to or attached about the second float stem portion 104 such that the inner diameter of the float collar 112 may be sized to match the outer diameter of the second float stem portion 104 or may have an inner diameter which is slighter smaller than the outer diameter of the second float stem portion 104. The length of the float collar 112 may also be relatively less than the length of the second float stem portion 104 such that the second float stem portion 104 may extend past the float collar 112 when the float collar 112 is secured upon the second float stem portion 104. Other variations may include a float collar 112 having a length which is equivalent to the length of the second float stem portion 104 or a length which extends past that of the second float stem portion 104. In either case, with the inner diameter of the float collar 112 being less than the outer diameter of the first float stem portion 102, the float collar 112 may be positioned over the second float stem portion 104 to abut the shoulder defined where the distal end of the first float stem portion 102 is attached to the proximal end of the second float stem portion 104.

In other variations, the foam collar may instead be positioned along the wide float stem portion while seated into secure placement within a circumferential groove defined around the float.

While the float collar 112 may be comprised of a foam material, the float collar 112 may be comprised of a number of different compressible materials capable of performing the functions described herein and is not limited to foam materials. Examples of some materials suitable for fabricating a foam float collar may include, for instance, silicone (which is particularly compatible with platelets and has a high permeability to oxygen and nitrogen), polyethylene, fluoroelastomer (e.g., VITON, The Chemours Company, DE), or a number of other polymers having sufficient shape memory.

Furthermore, the outer diameter of the float collar 112 may extend past the outer diameter of the first float stem portion 102 so that the outer diameter of the float collar 112 may contact the inner diameter of the tube or container 120 within which float 100 is positioned, as shown in the side view of FIG. 1B. With the float channel 106 defined through the float 100, any air trapped within the container 120 may escape through the float channel 106 as the float 100 is pushed into the container. In yet other alternative variations, the float may define a notch or passageway along an outer surface of the first float stem portion 102 to allow for air to escape from a lower portion of the container as the float 100 is advanced into the container during assembly.

As previously described, the float 100′ may be configured to incorporate a second float stem portion 130 which may be shaped into other structures. FIGS. 1C and 1D show respective side and bottom views of one example where the second float stem portion 130 is configured in a domed or rounded structure. The float collar 112 may be coupled to a proximal portion of the second float stem portion 130 adjacent to the distal end of the first float stem portion 102 so that the domed or rounded structure extends beyond the float collar 112. The shape of the domed or rounded structure may present an atraumatic surface which may optionally correspond to the interior bottom surface of the container 120.

The float 100′ with the collar 112 may be inserted into the interior of the container 120 such that the float 100′ forms an airtight seal between the float 100′ and the container interior, as illustrated in FIG. 1E. The float 100′ may incorporate a float channel 106, as previously described, or may incorporate a channel or notch along an outer surface of the float 100′ to allow for air trapped beneath the float 100′ to escape from the container interior and enable the float 100′ to be urged towards the bottom of the interior, as shown, during assembly.

FIG. 1F illustrates an example of a container having the float 100′ with the rounded second float stem portion 130 positioned within the interior of the container 120. A sample of blood is shown after processing in which the container 120 and sample have been spun to separate the components of the blood sample. The float 100′ and float collar 112 may be seen at an equilibrium position relative to the separated components.

In yet another variation, the float collar may be fabricated in another manner. One variation may include a float collar 140 formed as an O-ring where the float collar may be define a hollow interior 142 which may be filled with a gas (e.g., air, nitrogen, etc.) or liquid (e.g., water, saline, etc.). The float collar 140 may be formed of an elastomeric material, e.g., silicone, in various sizes for different float diameters. FIG. 1G illustrates an example of an O-ring float collar 140 having a circular cross-sectional shape when in its uncompressed configuration. The example illustrated shows the float collar 140 positioned upon a surface of the float stem portion 104 where the float collar 140 may be slightly compressed or compressible when subjected to centrifugally-generated pressures. The gas or liquid within the interior 142 may resist full compression yet provide a compressible collar sufficient to resist movement against a container interior wall when at rest within a container. While a gas may be compressible within the interior 142, the presence of a liquid may instead reconfigure the float collar 140 to a flattened configuration where the float collar 140 may expand in height while flattening to accommodate the redistribution of the fluid within.

Other variations of the float collar may include one having an elongated (e.g., elliptical, etc.) cross-sectional shape to increase the contact area between the float collar and the inner wall of the container. Another variation may include a rectangularly-shaped float collar 150 also defining a hollow interior 152, as shown in FIG. 1H. Such a rectangularly-shaped float collar 150 may provide an elongated surface area for contacting against the surface of the float stem portion as well as the interior of the container within which the float is placed.

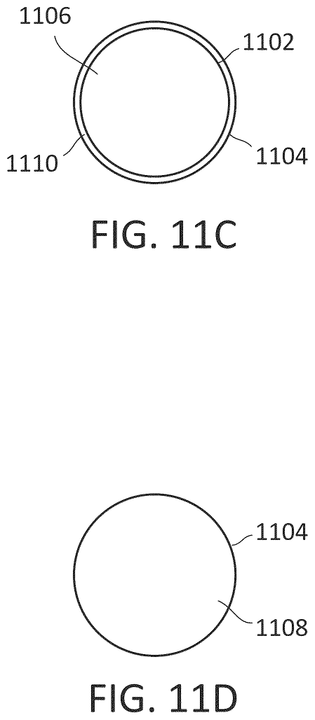

The float collar itself may be configured in any one of several different variations depending upon the desired configuration of the float collar. In one variation, the float collar may be fabricated to have all of its exposed surfaces to be smooth (or skinned) where the exposed surfaces may be processed to present a smooth surface. FIGS. 11 and 1J show top and bottom perspective views of float collar 150 where all exposed surfaces, .e.g., inner, outer, upper, and lower, have been smoothed (or skinned). Such a float collar 150 may be fabricated by molding to form the smooth surfaces. The smooth outer surfaces may provide for increased friction between the outer surface of the collar and the inner wall of the container to thereby enhance the lock of the float within the container, e.g., during shipping and handling.

In cases where the foam collar is fabricated from a closed cell foam material, the outer surfaces of the fabricated foam collar may be relatively rough where sliced-open cells are exposed. FIGS. 1K and 1L show top and bottom perspective views of float collar 152 where all of the exposed surfaces present relatively rough interface surfaces where the frictional force generated may be relatively lower than the frictional force generated between a smooth surface and the container.

Another variation is shown in the top and bottom perspective views of FIGS. 1M and 1N where the inner and outer surfaces of the float collar 154 are smooth but the upper and lower surfaces are exposed and relatively rougher. Such a float collar 154 may be fabricated, e.g., by cutting the float collar 154 from an extruded length of tubing in which case the upper and lower surfaces, as shown, may present the exposed sliced-open cells.

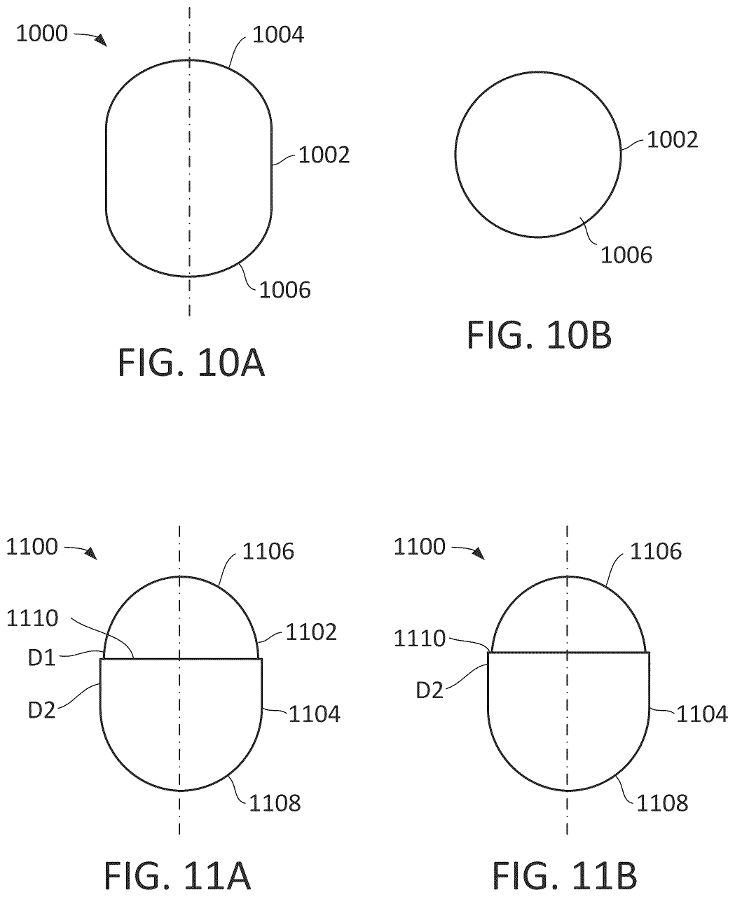

Another variation is shown in the top and bottom perspective views of FIGS. 10 and 1P of float collar 156 where the inner and outer surfaces present rough surfaces but the upper and lower surfaces are relatively smooth. A float collar 156 having smooth upper and lower surfaces may exhibit a lower tendency to trap cells within exposed pockets and a rough inner surface may facilitate the escape of gases (such as air) between the gaps formed by the rough surface and the float stem thereby obviating the need to provide a slot or hole for venting during assembly. Such a float collar 156 may be fabricated, e.g., by punching the float collar from a sheet stock in which case the inner and outer surfaces may be rough and the upper and lower surfaces either smooth or skinned depending on the nature of the sheet stock.

In any of these variations, only select surfaces may instead be roughened or smoothed depending upon the desired configuration. For instance, a float collar may be configured to present a smooth outer surface only while the other surfaces (inner, upper, and lower) may remain in a rough state. Such a float collar may be fabricated, e.g., by slicing a length of cord stock and punching a hole in the center in which case only the outer surface may be smooth.

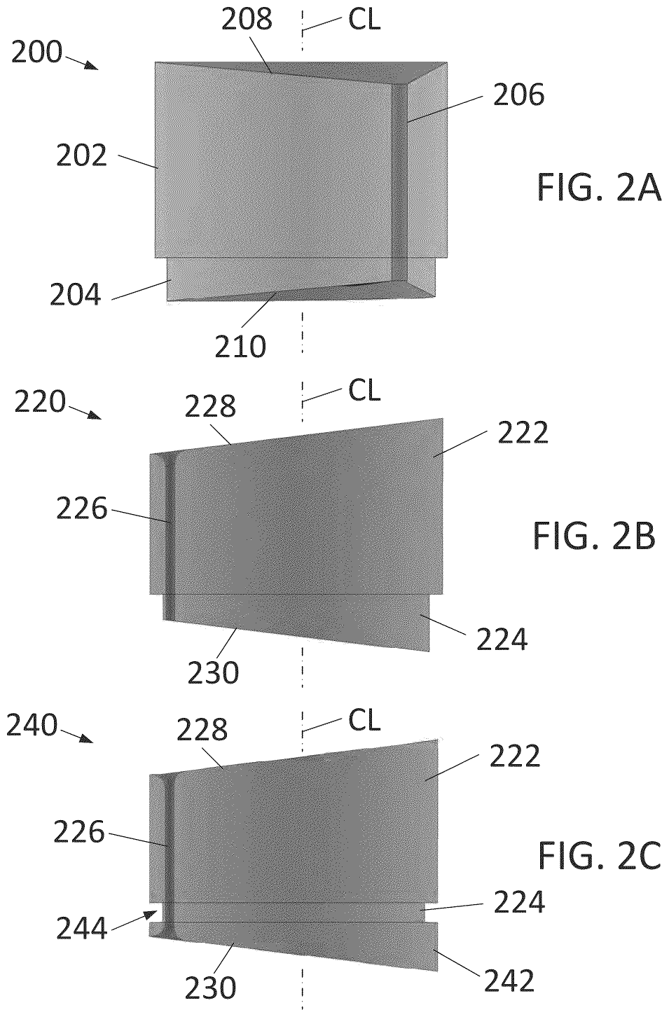

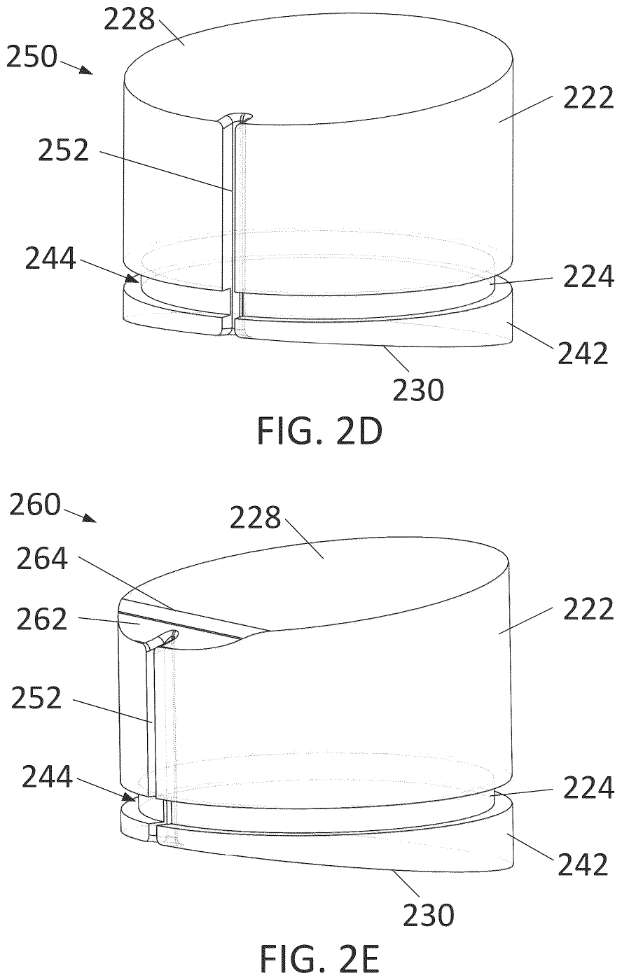

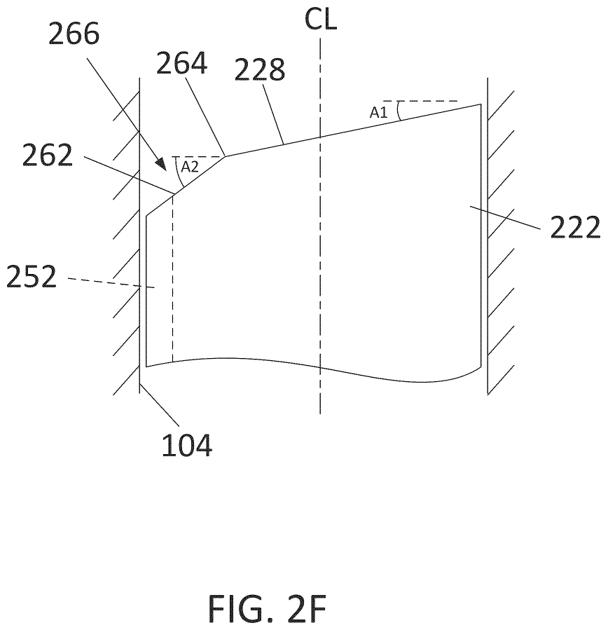

The float may be configured in yet other variations as shown in the side and perspective views of FIGS. 2A to 2E. The float may incorporate any of the float collar variations in any of these float embodiments as well and the float collar has been omitted from these figures only for clarity purposes.

FIG. 2A shows one variation where the float 200 may have a first float stem portion 202 with a first diameter and a second float stem portion 204 extending from a distal end of the first float stem portion 202 at a distance and having a second diameter which is less than the first diameter. The upper float portion 208 may forms a concave surface which is angled to form an indentation or acentric concavity where its lowest relative point has an opening in fluid communication with a float channel 206 extending through the interior of the float 200 from the upper float portion 208 to a lower float portion 210 which may also form an opening in its concave surface which is angled to form an indentation or acentric concavity where its opening is coincident with the float channel 206. The float channel 206 may extend through the float 200 at a location separated from the centerline CL of the float 200 such that the concave upper float portion 208 and concave lower float portion 210 form their respective concavities which are angled towards one another. While the float channel 206 is shown to extend in parallel with the centerline CL of the float 200, the float channel 206 may be angled or curved while extending between the upper float portion 208 and lower float portion 210 in which case the openings in each respective float portion 208, 210 may be repositioned to coincide with the float channel 206.

FIG. 2B shows a side view of another variation where the float 220 similarly includes a first float stem portion 222 having a first diameter and a second float stem portion 224 having a second diameter where the second diameter is less than the first diameter. The upper float portion 228 may define a first surface which is angled relative to the centerline CL of the float 220 such that the angled surface slopes entirely over the upper float portion 228. The surface may extend from a relatively lower end to a relatively higher end such that the lower end may define an opening in fluid communication with a float channel 226 which may extend in parallel through the first float stem portion 222 and the second float stem portion 224 to an opening defined along the lower float stem portion 230. The lower float stem portion 230 may similarly define a second surface which is angled relative to the centerline CL of the float 220 such that the angled surface slopes entirely over the lower float portion 230 in a direction opposite to the angle of the upper float portion 228 such that a height of the float 220 is at a minimum where the upper float portion 228 and lower float portion 230 are angled toward one another and the height of the float 220 is at a maximum where the upper float portion 228 and lower float portion 230 are angled away from one another.

The float channel 226 extend between the upper float portion 228 and lower float portion 230 near or at the minimum height of the float 220 such that the float channel 226 may extend through both the first float stem portion 222 and the second float stem portion 224. With this configuration, the white blood cells may slide or move over the sloped lower float portion 230 to percolate up and through the float channel 226 to the upper float portion 228 as the red blood cells sediment below the lower float portion 230 during usc. The buffy coat may form at the lower angled portion of the upper float portion 228.

FIG. 2C shows a side view of yet another variation where the float 240 similarly includes a first float stem portion 222 having a first diameter and a second float stem portion 224 having a second diameter where the second diameter is less than the first diameter. A third float stem portion 242 may be positioned distal to the second float stem portion 224 and have a third diameter which is equal to or similar to the first diameter. The second float stem portion 224 may form a channel or groove 244 which extends around a circumference of the float 240 within which any embodiment of the float collar described herein may be seated securely. Similar to the embodiment described above, the third float stem portion 242 may define the lower float portion 230 configured at an angle similarly as described above for FIG. 2B.

FIG. 2D shows a perspective view of yet another float variation 250 similar to the embodiment shown above in FIG. 2C. In this variation, the float channel 252 is formed as a channel or groove which remains open over its entire length along the side of the float 250 as it extends between the upper float portion 228 and the lower float portion 230 rather than extending through the first float stem portion 222 and the second float stem portion 224 as an enclosed channel. In this embodiment, the white blood cells may slide or move over the sloped lower float portion 230 to percolate up through the float channel 252 and along the interior wall of the container to the upper float portion 228.

FIG. 2E shows a perspective view of yet another float variation 260 similar to the embodiment shown above in FIG. 2D but in this embodiment, the upper float portion 228 may further include a second angled surface 262 having a second sloped angle A2 which is greater than a first sloped angle A1 of the upper float portion 228 relative to the centerline of the float 260, as shown in the partial cross-sectional side view of FIG. 2F. The second angled surface 262 may extend from a boundary 264 along the upper float portion 228 in proximity to the float channel 252 where the boundary 264 may extend along the surface of the upper float portion 228 perpendicularly relative to the direction of the slope of the upper float portion 228 and relative to the opening of the float channel 252. The second angle A2 of the second angled surface 262 may be relatively greater than the first angle A1 of the upper float surface 228 such that the second angled surface 262 may form a buffy coat collection region 266 against the inner wall of the container 104 when the float 260 is in use.

This collection region 266, as shown in FIG. 2F, may decrease the red blood cell presence in this collection region and further decrease the variability of the red blood cell level within the collection region as the equilibrium position of the float 260 will be influenced by the plasma density, which varies from patient-to-patient. The shift in the volume of the body of the float 260 located above the buffy coat position at equilibrium may generally result in a relatively smaller shift in the volume of fluid within the narrower collection area in which the buffy coat forms. This same feature can be included in any of the other float variations described herein. For example, a float having a concave upper float portion may have an increased indention at the base of the concavity.

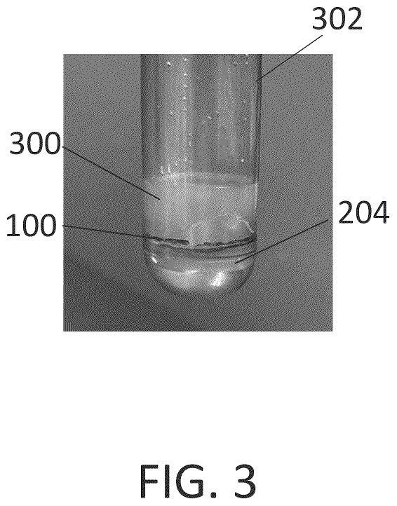

One embodiment for securing the float 100 within a container, e.g., for manufacturing, handling, and/or shipment of the float 100 and container, may include simply positioning the float 100 within the empty container. Another variation may include placement of the float 100 within the container with an amount of a fluid such as anticoagulant introduced above the float 100 to create a locking or securement feature through the formation of a vapor lock. FIG. 3 discloses a foam float collar 204 which is maintained in its position within a container 302 with at least a partial vapor lock. The foam collar float 100 may be pushed into the bottom of the container tube while displacing any air beneath the float 100 and a volume of fluid 300 such as an anticoagulant may be introduced into the container tube after inserting the float 100 until the top of the float 100 is covered by the fluid 300. The float collar 204 may be seen in contact against the inner surface of the container with the fluid 300 as shown above the float 100. The presence of the fluid 300 above the float 100 within the container creates a vapor lock as the air and/or fluid below the float is forced out of the lower portion of the container below the float 100 and the fluid 300 remains above the float 100 and inhibits the reintroduction of any air below the float 100 to prevent dislodgement or any further movement thereby locking the float 100 in position within the container. The vapor lock may be considered a partial vapor lock when at least a majority the air and/or fluid is expressed from below the float, e.g., through a slot in the side of the float or through the float channel 106. When the container tube meets a large amount of kinetic force, for example by banging the container tube against another solid object, the foam collar float 100 which was pushed down into the container tube without a partial vapor lock may be moved slightly relative to the tube floor. In contrast, when the container tube meets a large amount of kinetic force, for example by banging the container tube against another solid object, the foam collar float 100 which was pushed down into the container tube with a partial vapor lock may move an imperceptible distance relative to the floor.

While the fluid 302 introduced into the bottom of the container interior may be anticoagulant, other variations may instead use an isotonic saline as the fluid to form the vapor lock in order to secure a position of the float 100 at the bottom of the container. Any number of coagulants such as ACD-A anticoagulant (citrate) (Zimmer Biomet, IN) may be used where a small volume may be introduced into the container so that it can wick in and around the gap between the container interior wall and the float 100. The anticoagulant need not fill the entire gap from the bottom of the float to the top of the float so long as some amount of the liquid surrounds the entire circumference of the float 100 sufficient to create a liquid seal to prevent the dislodgment of the float 100 relative to the container. The sample to be processed may be simply introduced into the container with the anticoagulant already present for processing. A ratio of about 6 cc of ACD-A per 54 cc of blood may be sufficient to create a vapor lock although this ratio may be varied depending upon various factors, e.g., float size, container volume, sample volume, etc.

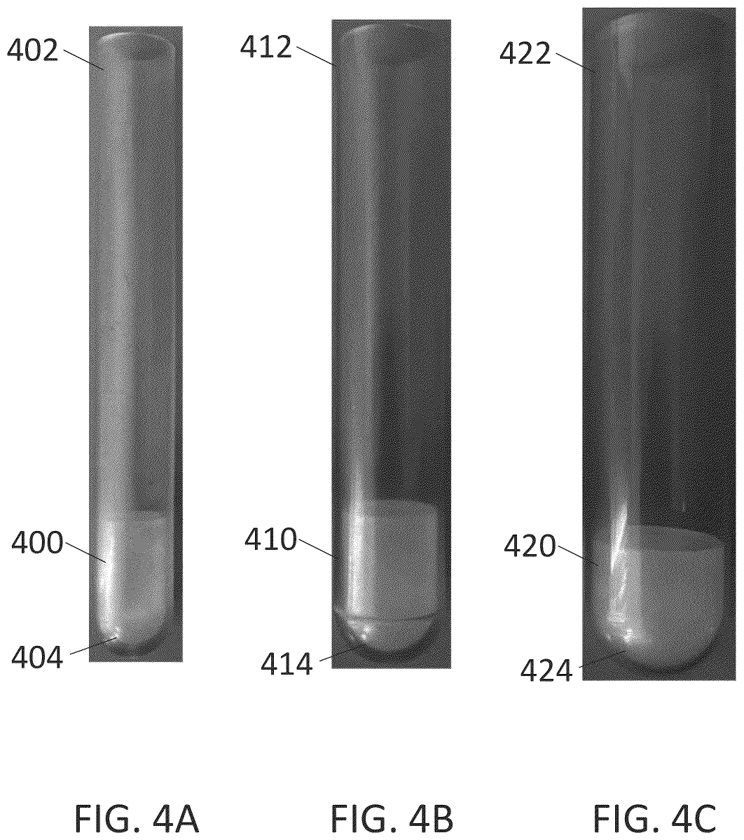

In yet another variation, FIGS. 4A to 4C illustrate side views of another float variation which may take advantage of the vapor lock securement for various float sizes. Because the vapor lock utilizes the pressure differential to secure a position of the float within the container, the float collar may be optionally omitted entirely from the float structure. The variation illustrated shows a several different float sizes (optionally omitting the float collar) for use in several different container sizes.

FIG. 4A illustrates a side view where float 400 is secured within container 402 at the bottom 404 of the container 402 which may contain volume of fluid such as an anticoagulant. The variation illustrates how a 12-cc container 402 having a tube inner diameter (ID) of about 12.9 mm may use a float 400 having an outer diameter (OD) of about 12.7 mm and a float length of about 25 mm.

FIG. 4B similarly illustrates a side view where float 410 is secured within container 412 at the bottom 414 of the container 412. The variation illustrates how a 22-cc container 412 having a tube inner diameter (ID) of about 19.3 mm may use a float 410 having an outer diameter (OD) of about 19.2 mm and a float length of about 24 mm.

FIG. 4C similarly illustrates a side view where float 420 is secured within container 422 at the bottom 424 of the container 422. The variation illustrates how a 50-cc container 422 having a tube inner diameter (ID) of about 26.4 mm may use a float 420 having an outer diameter (OD) of about 26.2 mm and a float length of about 27 mm.

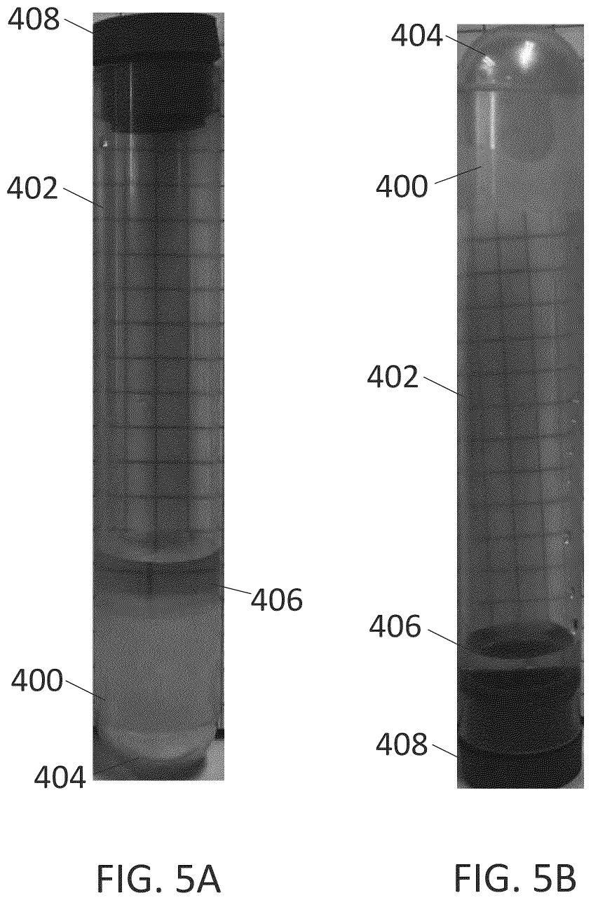

In each of these variations, the volume of fluid used to form the vapor lock for securing the float position may be enough to cover the float and prevent air from being reintroduced below the float after the float is initially pushed into the lower portion of the container. This vapor lock feature is shown in further detail in the side view of FIG. 5A which illustrates the float 400 pushed down into the bottom of the container 402 such that the air below the float 400 is displaced by the float 400. A volume of fluid 406 (e.g., anticoagulant) may then be introduced until the float is covered and the fluid 406 may pool and remain above the float 400. The container 402 may be sealed (e.g., optionally under vacuum) with a septum or covering 408, as shown, with the position of the float 400 secured to the bottom 404 of the container via the vapor lock. FIG. 5B illustrates how the float 400 may remain secured in place relative to the bottom 404 of the container 402 even when the container 402 is inverted such that the pooled fluid 406 falls away from the float 400. While this example illustrates the float omitting the float collar, the float collar may be optionally incorporated if so desired.

The use of the vapor lock may be omitted entirely and the float channel 106 may instead be simply sealed or obstructed after the float 100 has been pushed into the container or tube during assembly. The volume of air beneath the float 100 may be urged or forced out of the container through the float channel 106 as the float 100 is pushed into the container. Once the float 100 has been desirably situation within the float, the float channel 106 may be obstructed or sealed to maintain the position of the float 100 within the container for shipment purposes. As the float 100 moves away from the floor of the container, the presence of the fluid and the movement of the float 100 may form a negative pressure beneath the float 100 and prevent its movement thereby locking its position within the container. Furthermore, the outer diameter of the float collar 112 when uncompressed may be slightly larger than the inner diameter of the container. As the float is inserted within the interior of the container, the float collar 112 may compress and provide a radial force against the interior walls of the container to further secure the float in position relative to container. Once the float 100 is desired to be removed from the container, the float channel 106 may be unsealed or unobstructed to allow for air or fluid to be introduced into the container and may further allow the float to move to its equilibrium position before, during, and/or after processing.

In yet another variation, rather than implementing a vapor lock with a fluid, the interior of the container may instead be placed under vacuum and sealed via a septum or other cover or cap with the float contained within. The float position within the container may be maintained due to the radial force imparted by the float collar against the interior walls of the container and prior to use. The negative pressure within the container may also help to distort or deflect the walls of the container inwardly (for non-glass containers) to further help maintain a position of the float within although the radial force provided by the float collar may be sufficient to maintain the position of the float without the use of the vacuum.

However, the vacuum may serve an additional purpose to generate a platelet-rich fibrin matrix (PRFM) which is PRP produced by processing freshly drawn blood without anticoagulant. When the needle of a blood-filled syringe (or connected via tubing to a patient's vein) punctures the septum sealing the top of the tube, the vacuum within the tube may immediately draw or suck the blood into the tube due to the pressure difference between the tube interior and the blood-filled syringe. The container filled with the drawn blood and the float within may then be spun immediately to separate the PRP from the red cells and the PRP (now located above the float) may be quickly withdrawn from the container and used before the blood has time to clot.

During shipment and handling, the float position within the container may be maintained, as described; however, even if the float may have migrated away from the floor of the container, the float will drop to the bottom of the container as blood is introduced into the container from above.

In yet another alternative, the container interior may include a volume of fluid such as the anticoagulant and may also be placed under vacuum. As the float position is maintained, as described, by the vapor lock and the vacuum, the float may remain in its initial position at or near the bottom of the container even with the introduction of blood. Furthermore, with the float collar under vacuum, the float collar (particularly if fabricated from foam) may expand further securing the float position within the container. The float may remain in its position at the bottom and the fluid or anticoagulant located below the float may not initially mix with the blood until the container is processed by spinning in which case the anticoagulant may then mix with the blood as the float moves from its initial position to its equilibrium position between the component layers of the blood.

With the introduction of the blood into the container, the float collar may reduce further back to its initial state with the removal of the negative pressure. Depending on the air permeability of the polymer material, e.g., foam, comprising the float collar, the air in the open space or voids within the foam may bleed out into the vacuum resulting in the float collar relaxing over time to its original state as the pressure inside the voids equilibrates with the lower pressure in the container. With the introduction of the blood into the container, the vacuum within the container will be lost while the voids may remain at a lower relative pressure. Depending upon the resiliency of the polymer itself to recovery of the expanded state, the float collar may no longer expand to effectively maintain the float in place against the container wall. Yet these effects may generally be ignored as the interference between the float collar and interior walls of the container are sufficient to maintain the position of the float.

Furthermore, as the float collar expands to lock the float in position after centrifugation, there is a reduced chance of red cells becoming trapped beneath the float from contaminating the PRP during vigorous resuspension. However, the tight fit of the float within the container, even without a float collar, is sufficient to reduce any such risks. The float collar ensures that the float remains at the bottom of the container during shipping and handling.

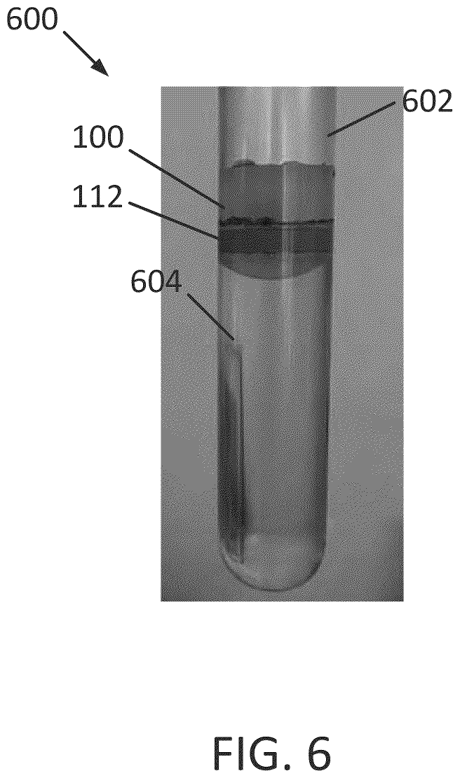

FIG. 6 discloses a foam collared float 100 at equilibrium after centrifuging within a volume of fluid 602 such as aqueous sucrose solution (e.g., having a density of about 1.1 gram per cubic centimeter) within a container tube 604 as an example resulting from a test performed to illustrate the effects of the density of the collar float 112 upon the performance of the float 100. The float 100 is shown as having a density of about 1.1 grams per cubic centimeter (similar to the density of RBC and plasma has a density of about 1.03 g/cc). The foam collar float 100 has a density of about 0.48 grams per cubic centimeter when squeezed between the second float stem portion 104 and the container tube wall and the overall density of the foam collared float 100 with the above dimensions was about 1.03 grams per cubic centimeter, where the float body has a density of about 1.08 grams per cubic centimeter. Fully compressed, the density of the foam collar 112 may be similar to that of silicone, e.g., between 0.95 and 1.2 grams per cubic centimeter, so the overall density of the foam collared float 100 may be between about 1.03 and 1.08 grams per cubic centimeter (or higher with a dense silicone). At pressures of about 40 psi when spinning, the foam will be less than fully compressed, and the compression may also influenced by the durometer and properties of the foam. In one variation, the foam can be Soma Foama 25 (Smooth-On, Inc., PA) which is a two-component platinum silicone casting foam. However, other foam materials or similarly compressible materials may also be used in other alternative variations.

The float 100 is shown to have not risen to the top of the volume of fluid 602 because the centrifugally generated pressure on the foam collar 112 diminishes as the radius of rotation at which the float 100 is rotated decreases. At some point, the centrifugally generated pressure may become insufficient to compress the foam collar 112 enough to prevent it from adhering against the inner wall of the container. Hence, the overall density of the float assembly 100 is ideally such that the float 100 remains at the bottom of the container 604 when spinning with whole blood and begins rises as the higher density red blood cell begin to pack within the container 604 and push the float 100 upwards above the red blood cell pack.

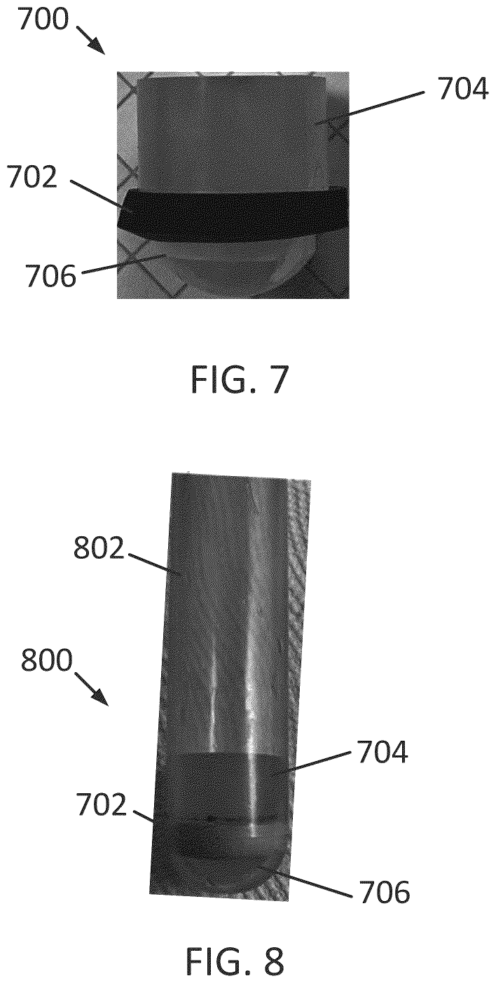

FIG. 7 is a perspective view of a foam collared float 700. The foam collared float 700 has a first float stem 704 and a second float stem 706 fixedly connected to the first float stem 704. The second float stem 706 is shown covered in this example. In one variation the first float stem 704 has a diameter of 0.758 inches and a length of 0.45 inches and the second float stem 706 a diameter of 0.635 inches and a length of 0.25 inches. In one variation the overall length of the foam collared float 700 is 0.832 inches including a rounded lower float portion 110 and the container tube has a diameter of 0.765 inches. The narrow float stem 704 is shown to have a sponge cord 702 wrapped around the outer portion of the narrow float stem 704. In one variation the sponge cord 702 has an inner diameter of 0.375 inches and an outer dimension of 0.625 inches, a length of 0.1875 inches, and a density of 0.33 grams per cubic centimeter uncompressed. In another variation, while the inner diameter of the container may be about 0.75 inches, the float collar may have an outer diameter which is slightly larger than 0.75 inches such that when the float and float collar are inserted into the container interior, the float collar may compress and provide a radial force to secure the float position relative to the container. FIG. 8 also shows a perspective view of the foam collared float maintained in position along an inner wall of a portion of a container tube 800. The container wall is shown partially removed for clarity purposes only. The sponge cord 702 is pressed between the second float stem 706 and the inner wall of the container tube 802 which sets the foam collar float 700 at a higher density due to the compression of the sponge cord 702. In one variation there is a scalable hole in the bottom of the tube or a scalable hole extending through the foam collared float 100.

With the foam collared float compressed against the inner wall of the container, the float may be maintained in position within the interior of the container. Despite movement of the container with the float positioned within, the float may maintain its relative position along the interior wall of the container. For instance, even with the assembly shown being struck multiple times against a surface, the position of the float within remains securely positioned along the wall of the container. Under centrifugation, the walls of the container may bulge or widen slightly allowing for the foam collared float 100 to rise to an equilibrium position with the bulk of RBC packing out beneath the foam collared float 100 and plasma above the foam collared float 100. Hence the foam collar float 100 may be configured to have its density tuned to automatically position itself between the two layers under centrifugation. For example, the foam collar float 100 may have a density which is tuned specifically for use with whole blood, e.g., 1000 to 1100 kg/m³ (or specific density of 1.0 to 1.1 grams per cubic centimeter at 25° C.), while in other variations, the foam collar float 100 may be fabricated to have a different density, e.g., 1.03 to 1.07 grams per cubic centimeter, etc.

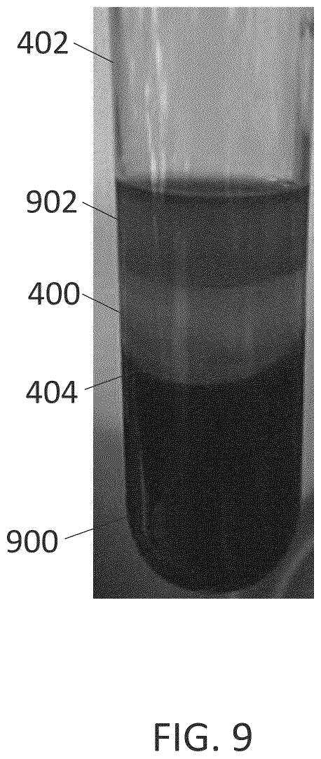

FIG. 9 shows a side view of a container 402 after processing a volume of whole blood. As shown, the float 400 (having omitted the float collar in this variation) may be seen having moved from a vapor lock secured position to an equilibrium position relative to the processed blood components. The float 400 is shown as having risen to a position above the packed red blood cells 900 with some of the platelet depleted plasma 902 removed before a buffy coat resuspension in order to increase the concentration of platelets and white blood cells in the harvested fraction after resuspension.

In yet another variation of the float a shown in the side and top views of FIGS. 10A and 10B, the float 1000 may be configured to have a dome or hemispherical shape defined on an upper portion 1004 of the float 1000 which extends from a cylindrically-shaped portion 1002. A lower portion 1006 of the float 1000 may also be dome or hemispherical shaped and may similarly extend from the cylindrically-shaped portion 1002. The upper portion 1004 may be defined as the portion which is positioned within the tube to face towards the opening of the tube while the lower portion 1006 may be defined as the portion which is positioned within the tube to face towards the enclosed bottom of the tube.

Both the upper portion 1004 and the lower portion 1006 may be symmetrically shaped relative to one another such that the curvature and radius of each portion 1004, 1006 are equivalent to one another while extending in opposite directions from each respective end of the cylindrically-shaped portion 1002. Alternatively, the upper portion 1004 may be configured to have a different height, curvature, or radius than the lower portion 1006.

In one variation, the float 1000 may have an upper portion 1004 and lower portion 1006 having a height of, e.g., 6.0-7.0 mm or 6.5-6.9 mm or 6.68 mm, and a radius of curvature of, e.g., 6.0-7.0 mm or 6.5-6.9 mm or 6.68 mm. The cylindrically-shaped portion 1002 may also vary in height ranging between, e.g., 5.0-6.0 mm or 5.0-5.5 mm or 5.2 mm, such that the diameter of the cylindrically-shaped portion 1002 may range between, e.g., 12.0-14.0 mm or 13.0-13.8 mm or 13.36 mm where the radius of the cylindrically-shaped portion 1002 may range between e.g., 6.0-7.0 mm or 6.5-6.9 mm or 6.68 mm.

In yet another variation of the float 1000, the dimensions may be increased to a relatively larger variation which may be positioned within a larger tube to accommodate larger volumes of blood. In one example the cylindrical portion may have a height of, e.g., 12.0-13.0 mm or 12.0-12.5 mm or 12.2 mm. The upper portion 1004 and the lower portion 1006 may each have a radius of, e.g., 13.0-14.0 mm or 13.0-13.5 mm or 13.2 mm, such that an overall height of the float 1000 may range between, e.g., 38.0-39.0 mm or 38.0-38.4 mm or 38.4 mm.

In either case, the lower portion 1006 may be configured to have a shape which corresponds to the shape of an interior bottom surface of the tubing into which the lower portion 1006 is placed so that the lower portion 1006 is able to mate with the curved, round interior bottom of the enclosing tube. With the lower portion 1006 mated against the bottom of the tube in a secure manner, a volume of anticoagulant (e.g., ACD-A anticoagulant) may be introduced into the tube so that the float 1000 remains covered by the anticoagulant when the tube is maintained in an upright position, particularly, for example, during storage, shipping, and handling.

In yet another variation of the float 1100, FIGS. 11A to 11D show side, top, and bottom views of a float 1100 having a dome or hemispherical shape defined on an upper portion 1106 of the float 1100 which extends from a first cylindrically-shaped portion 1102. A lower portion 1108 of the float 1100 may also be dome or hemispherical shaped and may similarly extend from a second cylindrically-shaped portion 1104 which is adjacent to the first cylindrically-shaped portion 1102. However, the first cylindrically-shaped portion 1102 may have a first diameter D1 ranging from, e.g., 13.0-13.3 mm or 13.26 mm where the radius of the first cylindrically-shaped portion 1002 may range between e.g., 6.5-6.65 mm or 6.63 mm, while the second cylindrically-shaped portion 1104 may have a second diameter D2 which is relatively larger than the first diameter D1 and ranging from, e.g., 12.0-14.0 mm or 13.0-13.8 mm or 13.36 mm where the radius of the second cylindrically-shaped portion 1004 may range between e.g., 6.0-7.0 mm or 6.5-6.9 mm or 6.68 mm. In this manner, a step or shoulder 1110 may be defined between the first and second cylindrically-shaped portions 1102, 1104, as shown in FIG. 11A.

Alternatively, the first cylindrically-shaped portion 1102 may be omitted entirely such that the second cylindrically-shaped portion 1104 may extend along the entire length to form a cylindrical portion having a single diameter along its length, as shown in FIG. 11B. The upper portion 1106 may terminate upon the cylindrically-shaped portion 1104 so that the reduced radius of the upper portion 1106 relative to the cylindrically-shaped portion may form the step or shoulder 1110. Hence, the smaller the relative radius of the upper portion 1106, the larger width of the resulting step or shoulder 1110.

In either case, the step or shoulder 1110 may be defined as an annular ring-shaped surface extending around the entirety of the float 1110 and can remain flattened or transverse relative to a longitudinal axis of the float 1110. In other variations, the step or should 1110 may be defined to extend partially around the float 1110 by some predetermined amount rather than around the entire periphery. Alternatively, the step or shoulder 1110 can instead define a slope or chamfer having an angled surface which presents a concave or convex like surface.

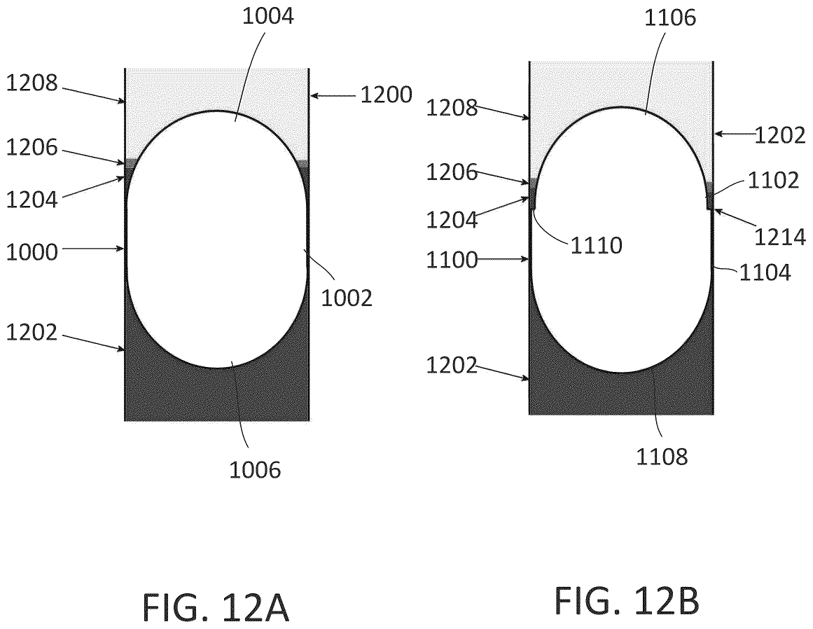

With either variation, a volume of whole blood may be introduced into the tube interior and over the float contained within. As the tube and blood sample is centrifuged, the whole blood will begin to separate out into its various components. FIG. 12A shows a side view of float 1000 contained within tube 1200 and the resulting separation of blood components. The upper portion 1004 guides the red blood cells (RBC) down the surface and past the cylindrical portion 1002 while the platelets and white blood cells (WBC) may also flow down the upper portion 1004 and mix with the platelets and WBC which have percolated up from beneath the float 1000 and past the cylindrical portion 1002 to form a ring of buffy coat or platelet-rich plasma (PRP) at equilibrium. With the upper portion 1004 configured into a dome or hemispherical shape, the layer of PRP 1206 containing both platelets and WBC is separated into the ring at equilibrium near the base of the dome of the upper portion 1004 while remaining separated by the cylindrical portion 1002 from the majority of the RBC 1202 located below the float 1000 and the lower portion 1006. While a small portion of RBC 1204 may remain above the cylindrical portion 1002, the RBC 1204 will remain below the PRP 1206 layer. The platelet-poor plasma (PPP) 1208 component may form a resulting layer located above the PRP 1206 layer, as shown.

While the overall density of the float may be varied between, e.g., 1.03-1.07 g/cc, the density of the float may be tuned to vary its resulting position, at equilibrium, relative to the various components of blood. For example, the density may be adjusted to ensure that the layer of PRP 1206 forms above the cylindrical portion 1002 near or at the base of the upper portion 1004.

With the PRP 1206 layer separated and isolated from the majority of the RBC 1202 layer the amount of RBC contaminating the PRP can be minimized as the platelets and WBC are concentrated within a ring-shaped layer and this may also decrease patient-to-patient performance variability.

The cylindrical portion 1002 along with the lower portion 1006 occupies a volume which, at equilibrium, displaces a volume of RBC greater than a volume occupied by part of the upper portion 1004 which sits in plasma above the layer of PRP 1206. This ensures that the equilibrium position of the float 1000 is influenced to a greater extent by the density of packed RBC than that of plasma. Because the variability of RBC density from patient to patient is less than that of plasma, this results in a lesser variation in the equilibrium position of the PRP 1206 layer relative to the float 1000. Thus the volume of RBC 1204 between the PRP 1206 layer and the base of the upper portion 1004 varies less from patient to patient and can be minimal.

FIG. 12B similarly shows a side view of float 1100 within tube 1200 and may function similarly to float 1000 in separating the various components of whole blood. The annular step or shoulder 1110 may function to help capture platelets and WBC in the PRP 1206 layer near or at the base of the upper portion 1106. With the difference in diameter between the first and second cylindrically-shaped portions 1102, 1104, the outer surface of the second cylindrically-shaped portion 1104 may contact against the inner surface of the tube 1200 while the recessed surface of the first cylindrically-shaped portion 1102 may be maintained at a distance from the inner surface of the tube 1200. The PRP 1206 layer may be retained within this recessed region above the step or shoulder 1110.

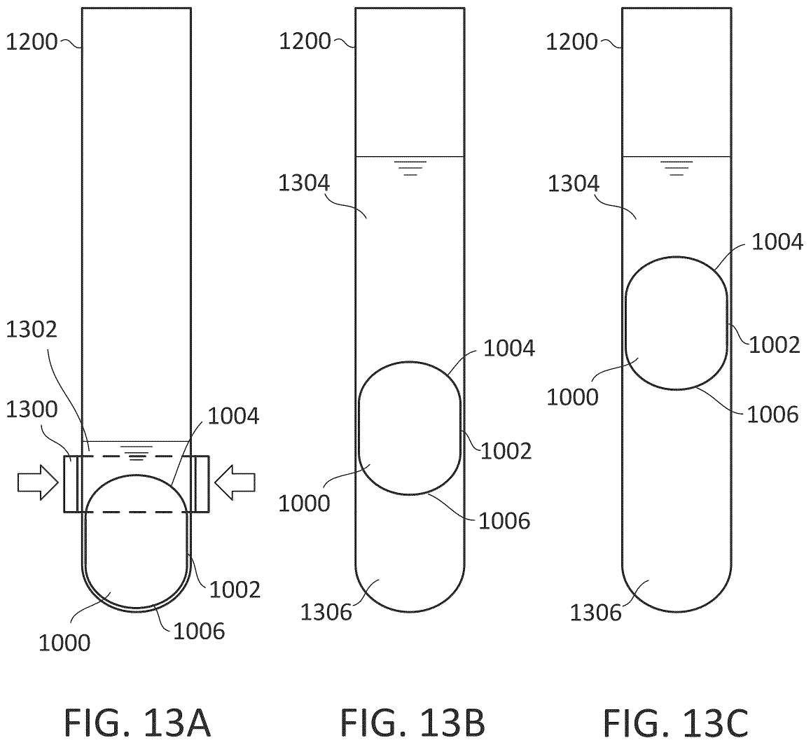

The float 1000 may be maintained within the bottom of tube 1200 such that lower portion 1006 of float 1000 is mated against the curved bottom surface 1306 of the tube 1200 in a corresponding manner. An external compression member 1300 such as a removable clamp, clip, band, etc. may be placed over the external surface of the tube 1200 in proximity to the float 1000 such that the external compression member 1300 compresses against the tube wall to temporarily flex the tube directly against the float 1000 to securely maintain a position of the float 1000 via direct compression or friction by preventing its movement relative to the tube 1200 interior, as described above, during storage, shipping, and handling. FIG. 13A illustrates how the positioning of the external compression member 1300 relative to the secured position of the float 1000 may be varied such that the position of the member 1300 is instead positioned to compress against the external surface of the tube 1200 directly above the securement location of the float 1000. For instance, the member 1300 may be positioned to compress directly above where the cylindrical portion 1002 of the float 1000 ends so that the lower portion 1006 of float 1000 is entrapped against the bottom 1306 of the tube 1200 and the float 1000 is prevented from migrating within the tube 1200. In this manner, the float 1000 position may be secured without directly compressing against the surface of the float 1000. A volume of anticoagulant 1302 may be optionally introduced over the float 1000 prior to storage and handling.

Prior to introducing whole blood into the tube 1200, the external compression member 1300 may be removed and the volume of blood 1304 may begin to mix with the anticoagulant 1302, as shown in FIG. 13B. As the tube 1200 undergoes centrifugation, the float 1000 may begin to rise within the tube 1200 as the various blood components begin to separate until full separation is achieved and the float 1000 reaches equilibrium, as described herein, and as shown in FIG. 13C.

As described above, the position of the float 1000 may be maintained within the tube 1200 by the use of an external compression member 1300 which may be removed from the tube. In other variations, the compression member may be integrated or formed directly into the packaging used for storing and/or shipping the tube so that the compression member may retain the float 1000 position within the tube 1200 as well as secure the tube 1200 itself for shipping and handling purposes. As the tube 1200 is removed from the packaging, the float 1000 position may be released automatically thereby omitting any removal of an external compression member.

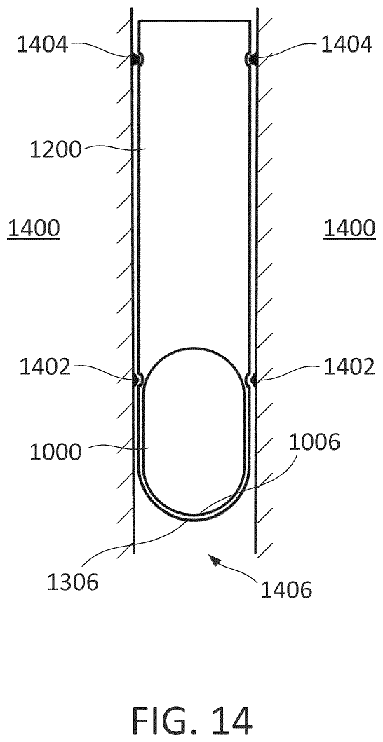

FIG. 14 illustrates an example of a packaging 1400, e.g., blister pack, in which a receiving channel or pocket 1406 is formed for retaining the tube 1200 within. The compression member 1402 may be formed as a restriction or narrowed portion 1402 in the sides of the receiving channel 1406 such that the restrictions 1402 extend transversely into the receiving channel 1406 to compress upon the external surface of the tube 1200. The restrictions 1402 may be formed to compress upon the external surface of the tube 1200 directly above the cylindrical portion of the float 1000 when the float 1000 is positioned within its securement location when the curved lower portion 1006 of the float 1000 is secured against the corresponding curved bottom 1306 of the tube 1200, as shown. One or more additional restrictions 1404 may be defined along the receiving channel 1406 to compress against the external surfaces of the tube 1200 to further provide a secure hold against the tube 1200 within the packaging 1400.

The tube 1200 may be sterilized directly when secured within the packaging 1400 through various sterilization methods. In one variation, gamma radiation may be used to sterilize the packaging 1400 and tube 1200. The gamma radiation may harden any plastic materials including the walls of the tube 1200 such that after removal of the tube 1200 from the packaging 1400, the walls of the tube 1200 may be not immediately straighten or relax back to its straightened configuration such that the float 1200 may still be retained within its securement location. However, even with radiation sterilization or long-term storage, the walls of tube 1200 will regain their straightened cylindrical form during centrifugation such that the float 1200 can freely move to its equilibrium position within the blood sample.

In these or any of the variations described, the tube 1200 may be evacuated and/or stoppered or sealed prior to, during, or after sterilization. For instance, the tube 1200 and float 1000 (and/or anticoagulant) positioned within the lumen of the tube 1200 may be evacuated and/or stoppered or sealed prior to securement within the packaging 1400. The entire assembly and packaging 1400 may then be sterilized although the order of scaling, securement, and sterilization may be altered in other variations and are intended to be within the scope of this disclosure.

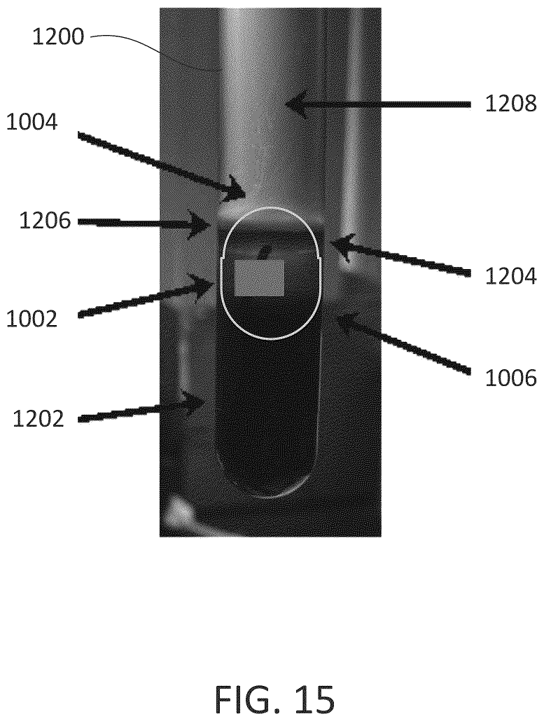

FIG. 15 shows a side view of another example of the separated blood components after centrifugation. As shown, the bulk of the layer of RBC 1202 may be seen separated and packed below the float, at equilibrium, within the tube 1200. The PRP 1206 layer may be seen above the cylindrical portion 1002 along with a relatively small volume of RBC 1204 at the base of the upper portion 1004. The lower portion 1006 of the float may also be seen extending down into the volume of RBC 1202 while the layer of PPP 1208 is shown above the PRP 1206 layer.

The variations of the floats may be used in any number of combinations with other features described throughout the description herein. For example, the various floats incorporating domed or hemispherical portions may be used with any of the other features described such as the use of a vapor lock or float channel in combination with the domed portions. In other examples, the floats incorporating domed or hemispherical portions may incorporate a float channel which can be defined to extend through or along a side of the float from the lower portion to the upper portion along with any other features described herein.

The apparatus and methods disclosed above are not limited to the individual embodiments which are shown or described but may include combinations which incorporate individual features between the different variations. Modification of the above-described assemblies and methods for carrying out the invention, combinations between different variations as practicable, and variations of aspects of the invention that are obvious to those of skill in the art are intended to be within the scope of the claims.

What is claimed is:

1. A float retention assembly, comprising:
a tube defining a lumen and a bottom end having a curved surface;
a float having a cylindrical portion with an upper portion and a lower portion each having a domed or hemispherical configuration and extending from opposite ends of the cylindrical portion, wherein the lower portion is configured to correspond in shape to the curved surface of the tube, and
wherein a density of the float is configured to position the cylindrical portion, at equilibrium, below a layer of platelet-rich plasma such that the platelet-rich plasma is retained between the upper portion and an interior surface of the lumen.

2. The assembly of claim 1 wherein the float further comprises a float channel defined through or along a side of the float from the lower portion to the upper portion.

3. The assembly of claim 1 wherein the cylindrical portion defines a first portion having a first diameter and a second portion having a second diameter where the second portion is adjacent to the first portion.

4. The assembly of claim 3 wherein the first diameter is less than the second diameter such that a step or shoulder is defined between the first portion and the second portion.

5. The assembly of claim 1 wherein a radius of the upper portion is less than a radius of the cylindrical portion such that a step or shoulder is defined between the upper portion and the cylindrical portion.

6. The assembly of claim 1 further comprising an external compression member positionable over an external surface of the tube in proximity to the float such that placement upon the external surface secures a position of the float within the lumen of the tube.

7. The assembly of claim 1 further comprising a volume of an anticoagulant within the bottom end of the tube.

8. The assembly of claim 7 wherein the anticoagulant comprises ACD-A anticoagulant.

9. The assembly of claim 1 wherein the lumen of the tube is evacuated.

10. The assembly of claim 9 wherein the lumen of the tube is sterilized.

11. A method of separating blood, comprising:
introducing a volume of blood into a tube defining a lumen and a bottom end having a curved surface;
applying a force to the blood contained within the tube and to a float positioned within the lumen, wherein the float has a cylindrical portion with an upper portion and a lower portion each having a domed or hemispherical configuration and extending from opposite ends of the cylindrical portion, and wherein the lower portion is configured to correspond in shape to the curved surface of the tube; and
further applying the force until the float reaches a position of equilibrium within the tube such that the cylindrical portion is positioned below a layer of platelet-rich plasma which is retained between the upper portion and an interior surface of the lumen.

12. The method of claim 11 wherein applying the force comprises applying a centrifugal force to the blood contained within the tube.

13. The method of claim 11 further comprising introducing a volume of anticoagulant within the bottom end of the tube prior to introducing the volume of blood.

14. The method of claim 13 wherein introducing the volume of anticoagulant comprises introducing ACD-A anticoagulant.

15. The method of claim 11 wherein the float further comprises a float channel defined through or along a side of the float from the lower portion to the upper portion.

16. The method of claim 11 wherein the cylindrical portion defines a first portion having a first diameter and a second portion having a second diameter where the second portion is adjacent to the first portion.

17. The method of claim 16 wherein the first diameter is less than the second diameter such that a step or shoulder is defined between the first portion and the second portion.

18. The method of claim 11 wherein a radius of the upper portion is less than a radius of the cylindrical portion such that a step or shoulder is defined between the upper portion and the cylindrical portion.

19. The method of claim 11 further comprising removing an external compression member from an external surface of the tube in proximity to the float prior to applying the force such that the float is movable within the lumen of the tube.

20. The method of claim 11 further comprising evacuating the tube prior to introducing the volume of blood.

21. The method of claim 20 further comprising applying an external compression member upon an external surface of the tube in proximity to the float prior such that the float is prevented from movement within the lumen of the tube.

22. The method of claim 21 further comprising sterilizing the tube and float.
